(19) United States
(12) Patent Application Publication
(10) Pub. No.: US 2025/0177777 A1
(43) Pub. Date: Jun. 5, 2025

(54) MULTI-CHANNEL RADIOACTIVE SOURCE IMPLANTATION DEVICE AND ITS METHOD OF USE

(71) Applicant: HANGZHOU DASHTECH CO., LTD, Hangzhou (CN)

(72) Inventors: Xuetang WANG, Hangzhou (CN); Dingchen ZHU, Hangzhou (CN); Guangming FU, Hangzhou (CN); Xingxing LEl, Hangzhou (CN)

(21) Appl. No.: 18/820,949

(22) Filed: Aug. 30, 2024

Related U.S. Application Data
(63) Continuation-in-part of application No. PCT/CN2023/078870, filed on Feb. 28, 2023.

(51) Int. Cl. A61N 5/10

(52) U.S. Cl. CPC A61N 5/1007; A61N 2005/1009; A61N 2005/1011

(57) ABSTRACT

This present invention discloses a multi-channel radioactive source implantation device and its method of use. The device comprises a main control body, a first motion platform, a first connecting portion, a wire output channel, a pushing wire, a wire driving mechanism, and a radioactive source feeding mechanism. The wire driving mechanism connects to the wire output channel. The radioactive source feeding mechanism is used to set radioactive source in the front of the pushing wire. The wire driving mechanism is used to drive the pushing wire to move back and forth along the wire output channel. The wire output channel is a rigid structure or a flexible structure that can be bent. One end of the wire output channel and the first connecting portion are respectively located on the opposite sides of the first motion platform. The first motion platform is used to control the relative motion in space between one end of the wire output channel and the first connection part, so that it makes the wire output channel to dock with one end of connection hole of different flexible delivery tubes, and the seeds or seed strands are pushed out from different connection holes, thereby achieving multi-channel implantation. This invention helps to enhance the implantation precision and efficiency, achieves fully automated operation, and helps to avoid radiation risks and reduce surgical time.

### CROSS REFERENCE TO RELATED APPLICATION

This application is a continuation of International Patent Application No. PCT/CN2023/078870, filed on Feb. 28, 2023, the entire contents of which are hereby incorporated by reference.

### TECHNICAL FIELD

The present invention relates generally to the field of medical device. More specifically, the present invention relates to a multi-channel radioactive source implantation device be used in radioactive source implantation surgeries, also includes its method of use.

### BACKGROUND TECHNOLOGY

Radioisotope seeds implantation is a technique which refers to directly implanting isotopic radioactive sources into tumor areas for treatment. It belongs to one of radiation therapies. Currently, this technique places radioactive sources into or around the tumor via interstitial implantation with guidance of imaging data provided by CT or ultrasound to. The radioactive sources continuously emit radiation to destroy the tumor cells. The implanted radioactive sources are typically iodine-125 seeds, with a half-life of 59.6 days and a radiation radius of less than 1.7 cm within the human body. So the seeds have the advantage of being safe and shielding easily. They emit gamma rays which effectively irradiate tumor cells for a continuous period of 180 days. This technique has the characteristic of high-dose distribution within tumor areas to kill tumor cells while surrounding normal tissues receive minimal radiation, causing little to no damage. Essentially, this technique is a form of precise radiotherapy.

Chinese Pub. No. CN1069415A, CN1069063C, CN1190602A, CN1322578A, and CN2235827Y disclose a treatment method and device suitable for various tumors in the human body. Before treatment, a tube or a needle connected with a tube is inserted into the tumor site in the human body, and the radioactive source is fixed to the end of a steel wire rope. It is then delivered through the tube to the tumor site for therapy. After treatment, the steel wire rope and radioactive source are retrieved. In such brachytherapy, the tip of the tube or puncture needle used is sealed (while in the seed implantation surgery, the tip of the hollow puncture needle is open for implanting seeds). And the puncture needle is connected to the front end of a flexible tube, and a radioactive source delivery device is installed at the rear end of the flexible tube. The radioactive source is delivered forward along the tube to the tumor (the radioactive source is not implanted into the body but emits radiation through the tube or the puncture needle). The radiation intensity of this radioactive source is much stronger than that of the I-125 seeds used in seed implantation surgery, achieving radiation therapy effects in just a few minutes. Compared to seed implantation surgery, this kind of therapy has a less irradiate time and cannot suppress tumor growth for an extended period. So, its efficacy in treating certain cancers is not as good as seed implantation surgery.

Yet, the seed implantation surgery requires manual intervention, which leads to the issue of doctors being exposed to radiation. Additionally, in the seed implantation surgery, the radioactive seeds need to be implanted into the body, so the implantation system need to overcome disinfection and biocompatibility challenges.

Chinese Pub. No. CN110496301A, CN 211214946U, WO2021022971A1 have disclosed a robot for targeted seed implantation in the lithotomy position suitable for clinical use. The robot comprises a frame, a pose adjustment mechanism, a friction wheel seed implantation device with tactile feedback, and a sine elastic amplification torque compensation mechanism. The use of a sine elastic amplification torque compensation mechanism allows for the compensation of gravitational torque in any arbitrary position of the arm, reducing the fluctuation of driving torque and improving the stability of the robot's low-speed operation at the end effector. However, the robot is rigidly connected to the puncture needle, which not only easily scratches the patient during the surgical process, posing a danger, but also leads to a complex and bulky connection structure at the tail end of the puncture needle. It limits the implantation location of radioisotope seeds, and increases the difficulty and duration of the surgical operation.

### SUMMARY OF THE INVENTION

In order to address the aforementioned technical issues, the objective of the present invention is to provide a multi-channel radioactive source implantation device and its method of use. The needle trocar connects to the radioactive source implantation device through a flexible delivery tube, which not only reduces the risk of scratching the patient but also enables multi-channel implantation. By setting up the first motion platform and first connection part, one end of a plurality of flexible delivery tubes are mounted on the first connection part, and one end of the wire output channel is mounted on the first motion platform. The first motion platform is used to achieve the relative motion in space between one end of the wire output channel and the first connection part, allowing the wire output channel to dock with any flexible delivery tube on the first connection part to form a delivery channel for seed or seed strand, thereby achieving multi-channel implantation.

To achieve the aforementioned objectives, this invention adopts the following technical solutions: A multi-channel radioactive source implantation device comprises a first motion platform, a first connecting portion, a wire output channel, a pushing wire, a wire driving mechanism, and a radioactive source feeding mechanism. The wire driving mechanism connects to the wire output channel. The radioactive source feeding mechanism is used to set up the radioactive source in front of the pushing wire. The wire driving mechanism is used to drive the pushing wire to move back and forth along the wire output channel. The wire output channel is a rigid structure or a flexible structure that can be bent. One end of the wire output channel and the first connecting portion are respectively located on the opposite sides of the first motion platform. The first motion platform is used to control the relative position in space of one end of the wire output channel and the first connecting portion.

Preferably, the first connecting portion connects to the first connection part, and the first connection part has a plurality of connection holes. One end of the flexible delivery tube connects on the corresponding connection hole, and at the other end of the flexible delivery tube is connected to a needle trocar. The first motion platform is used to achieve relative motion in space between one end of the wire output channel and the first connection part, so that it makes the wire output channel connects with each flexible delivery tube on the first connection part to form a delivery channel for the radioactive source, achieving multi-channel implantation. The first connecting portion is one or a combination of an adhesive connecting portion, a welding connecting portion, a threaded connecting portion, a snap-fit connecting portion, or a latch connecting portion.

Preferably, the first motion platform is one of the following modes:
A. The first connection part moves and one end of the wire output channel remains stationary.
B. The first connection part remains stationary and one end of the wire output channel moves.
C. The first connection part moves and one end of the wire output channel also moves.

The first motion platform is used to achieve the relative movement of at least two degrees of freedom between the first connection part and one end of the wire output channel. The mode of relative movement is one of the following:
A. The first connection part is stationary and one end of the wire output channel performs motion within a plane.
B. The first connection part performs linear motion back and forth and one end of the wire output channel performs motion within a plane.
C. The first connection part performs motion within a plane and one end of the wire output channel performs linear motion back and forth.
D. The first connection part performs linear motion back and forth and motion within a plane and one end of the wire output channel is stationary.

The motion within a plane is one of the following types: single rotational movement, a rotational movement combined with a radial linear movement, double-joint rotational movement, or two linear movements along the XY axes.

Preferably, the first motion platform includes a back-and-forth motion module, a rotation motion module, and a radial motion module. One end of the wire output channel and/or the first connection part achieves three degrees of freedom of movement in space via the rotational movement in one direction and the linear movements in two directions of the first motion platform.

Alternatively, the first motion platform includes a back-and-forth motion module, a left-and-right motion module, and an up-and-down motion module. One end of the wire output channel and/or the first connection part achieves three degrees of freedom of movement in space via the linear movements in three directions of the first motion platform.

Alternatively, the first motion platform is a multi-joint robotic arm, which can drive one end of the wire output channel and/or the first connection part to freely move and position in three-dimensional space.

Alternatively, the first motion platform includes a back-and-forth motion module, a rotation motion module. One end of the wire output channel and/or the first connection part achieves movement in space via the rotational movement in one direction and the linear movements in one direction of the first motion platform.

Preferably, the first motion platform is installed at the exterior of the main housing or within the interior of the main housing.

Preferably, when the first motion platform is installed inside the main housing and includes a back-and-forth motion module and a rotation motion module, the front side of the main housing is the first connecting portion, on which the first connection part is mounted. The first motion platform is designed in a drum-like shape, with the wire driving mechanism installed inside the drum. The rotation motion module drives the drum to rotate around its axis, and the back-and-forth motion module is installed inside the drum, used to drive the one end of the wire output channel to move back and forth.

Preferably, a combination of a wire driving mechanism, a magazine base, and a docking tube is installed in the first motion platform. The installation of the magazine base and docking tube is perpendicular to the first connecting portion. A magazine is mounted on the wire driving mechanism or on the magazine base.

When the first motion platform is installed with a wire driving mechanism and a magazine base, the wire driving mechanism and the magazine base are connected via a flexible tube. In this case, the flexible connecting tube is a part of the wire output channel, and the outlet of the magazine base is the front end of the wire output channel.

When the first motion platform is installed with a wire driving mechanism and a docking tube, the wire driving mechanism and the docking tube are connected via a flexible tube. In this case, the flexible tube is a part of the wire output channel, and the outlet of the docking tube is the front end of the wire output channel. The docking tube adopts a floating docking tube, which can float within a certain range.

Preferably, the flexible delivery tubes can be quickly installed onto the connection holes of the first connection part via quick connectors. There is a synchronized connector locking mechanism on the first connection part, which can simultaneously secure all the quick connectors mounted on the connection holes of the first connection part.

Preferably, the synchronized connector locking mechanism includes a connector clamping plate which is inside the first connection part. The connector clamping plate is equipped with connector clamping portions arranged in an array. In the unlocked state, all the connector clamping portions are staggered from the connection holes, allowing the quick connector of the flexible delivery tubes to be inserted into the connection holes without obstruction. During the locking process, the connector clamping portions are extended into the connection holes and inserted into the groove through the rotation or translation of the connector clamping plate driven by the clamping actuation mechanism, thereby securing all the quick connector of the flexible delivery tubes to the first connection part.

Preferably, the clamping actuation mechanism can be powered by electricity or manually operated, and it drives the rotation or translation of the connector clamping plate by a cam, a gear, or a belt drive.

Preferably, a flexible stylet is inside the flexible delivery tube, and it extends along the flexible delivery tube and fills the space of the needle trocar which is connected to the front end of the flexible delivery tube. This prevents blood from flowing into the needle trocar, otherwise blood coagulation can lead to the blockage of the needle trocar. The first connection part also has a synchronized stylet locking mechanism, it can simultaneously secure the flexible stylets which is inside each flexible delivery tube mounted on the first connection part.

Preferably, the synchronized stylet locking mechanism includes a stylet clamping plate which is inside the first connection part. The stylet clamping plate is equipped with stylet clamping portions arranged in an array. In the unlocked state, all stylet clamping portions are staggered with the connection holes, allowing the quick connector of the flexible delivery tube to be inserted through them. When locking the flexible stylet, a stylet clamping driving mechanism drives the stylet clamping plate to rotate or translate, misaligning the stylet clamping portions with the connection holes. The side of the quick connector of the flexible delivery tube has an opening that is connected to the internal elastic portion. The inside of the elastic portion adjacent to the flexible stylet inside this flexible delivery tube. As the stylet clamping plate rotates or translates, the stylet clamping portion presses against the elastic portion, so as to compress the flexible stylet through the elastic portion, achieving the flexible stylet locking.

Alternatively, the synchronized connector locking mechanism is the synchronized stylet locking mechanism. The connector clamping plate is the stylet clamping plate. The connector clamping portion is the locking stylet portion. And the clamping driving mechanism is the locking stylet driving mechanism. When the clamping driving mechanism drives the connector clamping plate to rotate by a first angle or to translate by a first distance, the connector clamping plate engages with the grooves of the quick connector of the flexible delivery tube without pressing against the elastic body. This action secures the quick connector of the flexible delivery tube to the first connection part, only achieving simultaneous locking and positioning of the quick connectors of all flexible delivery tubes, without simultaneously locking and positioning the flexible stylets inside the flexible delivery tubes. When the clamping driving mechanism drives the connector clamping plate to rotate by a second angle or to translate by a second distance, the connector clamping plate presses against the elastic body during the rotation or translation process. The flexible stylet is compressed by the elastic body, it achieves the simultaneous locking and positioning of both the quick connector and the flexible stylet inside each flexible delivery tube.

Preferably, before implantation, the flexible stylet must be pulled out. The flexible stylet is a flexible wire with elasticity, it can be bent under the effect of an external force and return to its straight state after the external force is removed. The length of the flexible stylet is greater than 600 mm. The rear end of the flexible stylet extends backward from the rear end of the flexible delivery tube. The distance between the front end of the flexible stylet and the front end of the needle trocar connected to the front end of the flexible delivery tube is not exceeding 5 mm.

Preferably, when the first motion platform is equipped with a wire driving mechanism and a docking tube, the first motion platform is used to achieve relative motion in space of the docking tube and the first connection part, allowing the docking tube to dock with one of the flexible delivery tubes on the first connection part. The docking tube includes a docking head and a first floating connecting mechanism. The docking head is mounted on the first motion platform via the first floating connecting mechanism which enables the docking head to float within a range.

Preferably, the first floating connecting mechanism adopts two rotational joint modules.

Alternatively, the first floating connecting mechanism adopts one rotational joint module and one translational module.

Alternatively, the first floating connecting mechanism adopts two translational modules, with the movement paths of the two translational modules being orthogonal to each other or at a certain angle.

Alternatively, the first floating connecting mechanism adopts a supporting base with an elastic ball hinge. The elastic ball hinge consists of a third elastic element and a hinge ball. The docking head is installed at the hinge ball which allows the docking head to pivot through a certain angle. The third elastic element is used to provide elastic force to reset the docking head when the docking head has been deflected.

Preferably, when the first floating connecting mechanism adopts two rotational joint modules, the first floating connecting mechanism includes a first link, a second link, and a link seat. The link seat is mounted on the first motion platform. The second link is rotatably set on the link seat through a first rotating shaft and achieves the rotational resetting via the first elastic element. The first link is rotatably set on the second link through a second rotating shaft and achieves the rotational resetting via the second elastic element. The docking head is set on the first link, or the docking head connects to the first link through a quick assembly and disassembly structure. The first rotating shaft is parallel to the second rotating shaft.

Preferably, the second elastic element is the first elastic element. One end of the first elastic element connects to the link seat, and the other end connects to the first link. While the first elastic element facilitates the rotational resetting of the first link, it also enables the rotational resetting of the second link.

Preferably, the radioactive source feeding mechanism acts as a cutting mechanism. The pushing wire itself is a seed strand or a seed strand casing, or the front half of the pushing wire is a seed strand or seed strand casing, and the rear half of the pushing wire is an elastic wire. The seed strand and seed strand casing can be cut by the cutting mechanism, and the target length of the seed strand or seed strand casing is severed from the front end of the pushing wire by the cutting mechanism, thereby achieving the feeding of the seed strand or seed strand casing. When the severed part is a seed strand casing, the radioactive source feeding mechanism also includes a seed embedding mechanism. This seed embedding mechanism is capable of inserting seeds and/or spacers into the seed strand casing from one end or the side, to form a complete seed strand. The cutting mechanism is positioned at any location along the wire output channel.

Alternatively, the radioactive source feeding mechanism uses a magazine for feeding. The radioactive source feeding mechanism is directly integrated into the wire output channel. Seeds, or seed strands, or seed strand casings are loaded into the storage slot or storage holes within the magazine. The feeding mechanism installed on the magazine is used to place seeds, or seed strands, or seed strand casings at the front end of the pushing wire for feeding.

When the magazine is loaded with a seed strand casing, the radioactive source feeding mechanism also includes a seed embedding mechanism. This seed embedding mechanism is capable of inserting seeds and/or spacers into the seed strand casing from one end or the side, forming a complete seed strand.

Alternatively, the radioactive source feeding mechanism uses a seed strand driving mechanism for feeding seed strands. The radioactive source feeding mechanism comprises a seed strand driving mechanism, a seed strand output channel, and a cutting mechanism. The radioactive source feeding mechanism outputs seed strands or seed strand casings by the seed strand driving mechanism and cuts them to the desired length by the cutting mechanism, to achieve the feeding of seed strands or seed strand casings. When the seed strand driving mechanism outputs a seed strand casing, the radioactive source feeding mechanism also includes a seed embedding mechanism. This seed embedding mechanism is capable of inserting seeds and/or spacers into the seed strand casing from one end or the side, thereby forming a complete seed strand. The seed strand driving mechanism is connected to the seed strand output channel. The seed strand output channel is a rigid structure or a flexible structure that can be bent. The seed strand output channel is connected with a divided channel or a channel switching mechanism. And the divided channel or the channel switching mechanism is also connected to the wire output channel.

The use method of the multi-channel radioactive source implantation device, including the following steps:
1) Connect a plurality of flexible delivery tubes to the corresponding connection holes on the first connection part, respectively. The other end of each flexible delivery tube connects to a needle trocar that is inserted into a target body.
2) Achieving the aligning of one end of the wire output channel with the different quick connector of the flexible delivery tube via the movement of the first motion platform. Then, establishing the docking between one end of the wire output channel and the quick connector of the flexible delivery tube via the back and forth movement of the first motion platform.
3) The wire driving mechanism drives the pushing wire to move back and forth along the wire output channel, which pushes the seed or seed strand forward. The seed or seed strand is set at the front end of the pushing wire by the radioactive source feeding mechanism. Then the seed or seed strand is delivered through the flexible delivery tube and implanted into the target body via the needle trocar connected at the front end of the flexible delivery tube.

Preferably, the radioactive source feeding mechanism adopts a magazine for supplying. The seeds or seed strands are set in the storage slot or storage holes of the magazine. The magazine is set with a fourth elastic element that pushes seeds or seed strands to the seed output channel that is connected with the wire output channel. When the wire driving mechanism drives the pushing wire to retract behind the magazine, the fourth elastic element pushes the seed or seed strand that is closest to the seed output channel within the storage slot or storage holes into the seed output channel, setting the seed or seed strand in front of the pushing wire. Then the wire driving mechanism drives the pushing wire to push the seed or seed strand in front of the pushing wire forward. The seed or seed strand is pushed out of the magazine. Subsequently, the wire driving mechanism drives the pushing wire to retract. The fourth elastic element uses its own elastic force to push the seed or seed strand that is closest to the wire output channel within the storage slot or storage holes into the wire output channel, positioning the seed or seed strand in front of the pushing wire. Subsequently, pushing the seed or seed strand out of the magazine. This operation is repeated until several seeds or seed strands have accumulated in front of the pushing wire.

Once, in step (2), one end of the wire output channel docks to one of the quick connectors of the flexible delivery tubes, the wire driving mechanism drives the pushing wire to push the accumulated seeds or seed strands in front of the pushing wire forward. It makes the seeds or seed strands to pass through the wire output channel, the flexible delivery tube, and the needle trocar, and to be implanted respectively into the target body, completing the implantation operation.

The present disclosure has the following beneficial effects compared with the prior art.

In the present invention, the needle trocar connects to the radioactive source implantation device through a flexible delivery tube, which not only reduces the risk of scratching the patient but also achieves multi-channel implantation. By setting up the first motion platform and the first connection part, one end of a plurality of delivery tubes are mounted on the first connection part. One end of the wire output channel is mounted on the first motion platform. The first motion platform is used to achieve the relative motion in space between one end of the wire output channel and the first connection part, allowing the wire output channel to dock with any delivery tube on the first connection part to form a delivery channel for seed or a seed strand, thereby achieving multi-channel implantation. Obviously, the structure is simple and well-designed.

In the present invention, during the seeds or seed strands implantation process, several radioactive sources needed for implantation via single needle trocar are pre-prepared according to the surgical requirements. It makes a plurality of seeds or seed strands to accumulate at the front of the pushing wire. Once the end of the wire output channel docks to different connection holes on the first connection part or the quick connector of the flexible delivery tubes, the wire driving mechanism drives the pushing wire to push the seeds or seed strands accumulated at the front of the pushing wire forward, through the wire output channel, the flexible delivery tube, and the needle trocar, until the seeds or seed strands are implanted into the target body, completing the implantation operation. It enhances surgical efficiency, achieves fully automated operation, and helps to avoid radiation risks.

### BRIEF DESCRIPTION OF THE DRAWINGS

The foregoing features and other aspects of the invention are explained in the following description taken in connection with the accompanying drawings wherein:

FIG. 1 is one of the structural diagram of seed implantation device of Embodiment 1.

FIG. 2 is another structural diagram of seed implantation device of Embodiment 1.

FIG. 3 is one of the structural diagrams of the first motion platform of Embodiment 1.

FIG. 4 is another structural diagram of the first motion platform of Embodiment 1.

FIG. 5 is one of the structural diagrams of a wire driving mechanism of Embodiment 1.

FIG. 6 is another structural diagram of a wire driving mechanism of Embodiment 1 (without one side plate).

FIG. 7 is another structural diagram of a wire driving mechanism of Embodiment 1.

FIG. 8 is a structural diagram of the seed implantation device with an isolation drape of Embodiment 1.

FIG. 9 is a structural diagram of first motion platform adopt three linear motion mechanism of Embodiment 1.

FIG. 10 is a structure diagram of Embodiment 2.

FIG. 11 is a structural diagram of the seed strand feeding mechanism of Embodiment 2.

FIG. 12 is an overall structural diagram of Embodiment 3.

FIG. 13 is a structural diagram of the magazine base of Embodiment 3.

FIG. 14 is a structural diagram of Embodiment 3 when the door of equipment housing is opened.

FIG. 15 is a structural diagram of the first motion platform of Embodiment 3

FIG. 16 is a structural diagram of the first connection part of Embodiment 3

FIG. 17 is an internal structural diagram of the first connection part of Embodiment 3.

FIG. 18 is a side-sectional view of the first connection part of Embodiment 3.

FIG. 19 is an enlarged structural diagram at position L in FIG. 18

FIG. 20 is a structural diagram of the first scheme of the synchronized connector locking mechanism and synchronized stylet locking mechanism in Embodiment 3.

FIG. 21 is a structural diagram of the second scheme of the synchronized connector locking mechanism and synchronized stylet locking mechanism in Embodiment 3.

FIG. 22 is a structural diagram of the synchronized connector locking mechanism and the synchronized stylet locking mechanism adopts translational movement of Embodiment 3.

FIG. 23 is a schematic diagram of the seed implantation into the target body in Embodiment 3.

FIG. 24 is a structural schematic diagram of the wheeled storage mechanism of Embodiment 3.

FIG. 25 is a structural diagram of the drum component in Embodiment 4.

FIG. 26 is a structural diagram of the docking tube structure and the needle pulling driver in Embodiment 4.

FIG. 27 is a structural diagram of the docking tube structure in Embodiment 4.

FIG. 28 is a structural diagram of the first floating connecting mechanism adopted two rotational joint modules of Embodiment 4.

FIG. 29 is a structural diagram of the first floating connecting mechanism adopt two rotational joint modules of Embodiment 4.

FIG. 30 is a structural diagram of the quick assembly and disassembly structure of the docking tube in Embodiment 4.

FIG. 31 is a structural diagram of the docking head of Embodiment 4.

FIG. 32 is a structural diagram of the second scheme of the first floating connecting mechanism.

FIG. 33 is a structural diagram of Embodiment 4 when the first link directly fixed with the docking head.

### DETAILED DESCRIPTION OF THE PREFERRED EMBODIMENT

The invention is further explained in combination with the drawings and embodiments.

The following provides a detailed description of the embodiments of the present invention, with examples of the embodiments being shown in the accompanying drawings. The examples described in reference to the drawings are illustrative and serve to explain the present invention, and should not be construed as limiting the scope of the invention.

### Embodiment 1

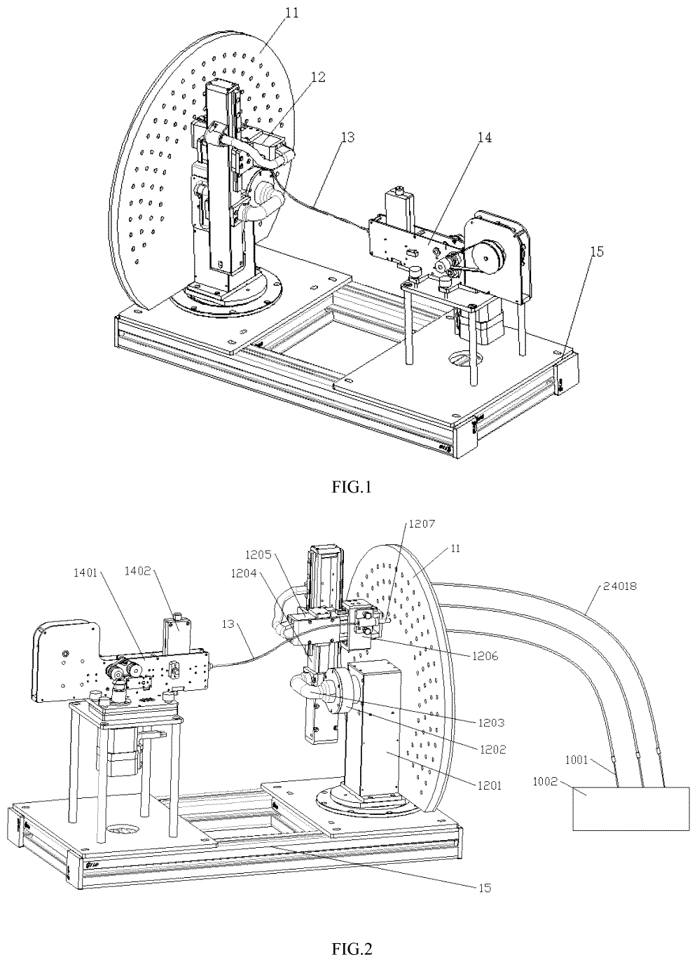

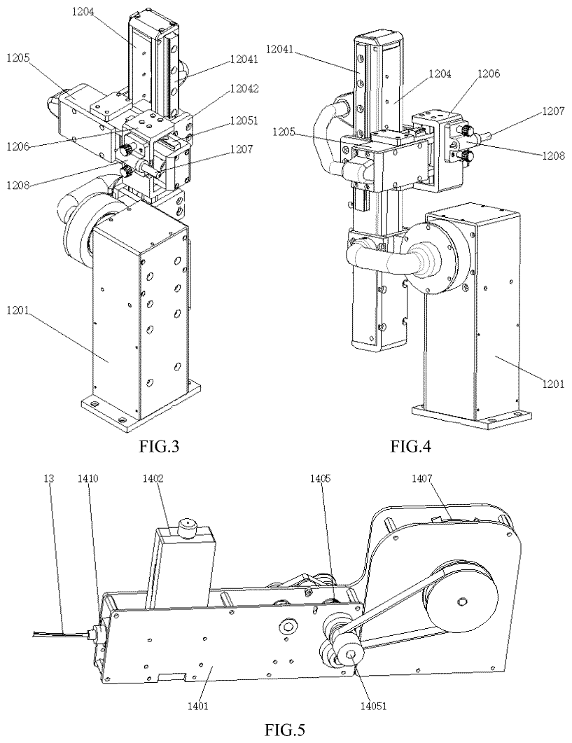

A multi-channel radioactive source implantation device illustrated in FIG. 1 and FIG. 2 comprises a main control body 15, a first motion platform 12, a first connecting portion, a wire output channel 13, a pushing wire, a wire driving mechanism 14, and a radioactive source feeding mechanism 1402. The radioactive source feeding mechanism 1402 is a seed magazine or a seed strand magazine. The wire driving mechanism connects to the wire output channel. The wire driving mechanism is used to drive the pushing wire to move back and forth along the wire output channel. The wire output channel is a rigid structure or a flexible structure that can be bent. One end of the wire output channel and the first connecting portion are respectively located on the opposite sides of the first motion platform. The first motion platform is used to control the relative position in space of one end of the wire output channel and the first connecting portion. The first connecting portion connects to the first connection part 11 which has a plurality of connection holes. One end of several flexible delivery tubes 24018 connect to corresponding connection holes, and at the other end, each flexible delivery tube 24018 connects to a needle trocar 1001. The first motion platform is used to achieve relative motion in space between one end of the wire output channel and the first connection part, so that the wire output channel docks with each flexible delivery tube 24018 on the first connection part to form a delivery channel for the radioactive source, achieving multi-channel implantation. The first connecting portion is one or a combination of an adhesive connecting portion, a welding connecting portion, a threaded connecting portion, a snap-fit connecting portion, or a latch connecting portion.

The first motion platform 12 and the wire driving mechanism 14 are mounted on the main control body 15. The wire output channel 13 is flexible and bendable. One end of the wire output channel 13 is mounted on one end of the first motion platform 12, and the other end connects to the wire driving mechanism 14. The first connection part 11 can be separately connects to a plurality of needle trocars 1001 via the corresponding flexible delivery tubes 24018. The needle trocar 1001 inserts into the target object 1002. The flexible delivery tube 24018 is the first flexible delivery tube. The first motion platform 12 is used to enable the movement of one end of the wire output channel 13 in two or three degrees of freedom in space, allowing the wire output channel 13 to dock with any one of the flexible delivery tubes. The radioactive source feeding mechanism 1402 is positioned at the front end (i.e., one end of the first motion platform 12) or the rear end (i.e., inside the wire driving mechanism 14) or the middle of the wire output channel 13, and it also facilitates the connection between the wire output channel 13 and the first connection part 11. A pushing wire is inside the wire output channel 13. The pushing wire is a flexible pushing wire 1301. The radioactive source feeding mechanism 1402 adopts a magazine for supplying radioisotope seeds or seed strands. The wire driving mechanism 14 comprises a seed pushing driving module that is used to drive the flexible pushing wire 1301. The seed pushing driving module pushes radioisotope seeds or seed strands out of the radioactive source feeding mechanism 1402, and pushes them along the first flexible delivery tube to the needle trocar.

As illustrated in FIG. 1 and FIG. 2, the first motion platform 12 performs a rotational movement in one direction and linear movement in two directions to achieve the movement of one end of the wire output channel 13 in three degrees of freedom in space. While the wire output channel 13 performs the guiding function for seed delivery, it also maintains its own flexibility to enhance the self-adaptability of the path during seed implantation. The wire driving mechanism 14 provides seeds and the force to deliver seeds, achieving seeds implantation. The first connection part 11 and the first motion platform 12 are mounted on the main control body 15 by a rotary joint, which provides a rotational degree of freedom. The wire driving mechanism 14 connects with the main control body 15. The first connection part 11 is used to dock with the wire output channel 13. The first connection part 11 can connect to the needle trocar through the first flexible delivery tube, thereby guiding the radioisotope seed through the wire output channel 13 and the first flexible delivery tube to be delivered to the needle trocar, until it is implanted into the tumor tissue.

In the present embodiment, the first motion platform 12 consists of three components: a back-and-forth motion module, a rotational motion module, and a radial motion module, achieving three degrees of freedom in movement.

Alternatively, the first motion platform can also perform linear motion in three directions as illustrated in FIG. 9. One end of the wire output channel and/or the first connection part achieves three degrees of freedom of movement in space via the linear movements in three directions of the first motion platform. The first motion platform 12 consists of three components: a back-and-forth motion module, a left-and-right motion module, and an up-and-down motion module, achieving three degrees of freedom in movement. Specifically, a seed implantation system which includes an up-and-down motion module 1, a left-and-right motion module 2, a back-and-forth motion module 3, a seed guidance module 4 (first connection part), a seed implantation applicator 5 (wire driving mechanism), and a main control body 6. The up-and-down motion module 1 is used to achieve the up-and-down movement of the seed implantation applicator 5. The left-and-right motion module 2 is used to achieve the left and right movement of the seed implantation applicator 5. The back-and-forth motion module 3 is used to achieve the back and forth movement of the seed implantation applicator 5. The first connection part 4 is used for guiding and mounting the flexible delivery tube. The front end of the flexible delivery tube is connected with a needle trocar inserted into a target body. The up-and-down motion module 1, the left-and-right motion module 2, and the back-and-forth motion module 3 change the position of the seed implantation applicator 5 to dock the seed implantation applicator 5 with each flexible delivery tube mounted on the seed guidance module 4, achieving multi-channel seed delivery and implantation.

Alternatively, the first motion platform is a multi-joint robotic arm, which can drive one end of the wire output channel 13 to freely move and position within three-dimensional space.

Alternatively, the first motion platform includes a back-and-forth motion module, and a rotation motion module. One end of the wire output channel and/or the first connection part performs relative movement in space via the rotational movement in one direction and the linear movement in one direction of the first motion platform. The rotational axis of the rotational movement is parallel to the direction of the linear movement.

As illustrated in FIG. 3 and FIG. 4, the back-and-forth motion module comprises the back-and-forth motion module main body 1205, the back-and-forth motion driving mechanism, and a docking tube connecting base 1206. The back-and-forth motion driving mechanism installs on the back-and-forth motion module main body 1205 which has back-and-forth motion rails 12051 installed on two sides. The docking tube connecting base 1206 slides along the back-and-forth motion rails 12051 with a slider, and the docking head 1207 installs on the docking tube connecting base 1206. The radial motion module comprises a radial motion module main body 1204 and a radial motion driving mechanism. The fixed end of the radial motion driving mechanism secures on the radial motion module main body 1204, and the movable end of the radial motion driving mechanism connects to the back-and-forth motion module main body 1205. This allows the radial motion of the back-and-forth motion module of the first motion platform to be achieved through the radial motion of the radial motion driving mechanism.

In the present embodiment, both the radial motion driving mechanism 1218 and the back-and-forth motion driving mechanism 1219 comprise one or more combinations of linear motors, gear rack mechanisms, lead screw and nut mechanisms, and belt drive mechanisms.

The rotational motion module comprises a first mounting base 1201 and a rotational driver. The first mounting base 1201 is installs on the main control body 15. And the rotating drive is fixed with the radial motion module main body 1204.

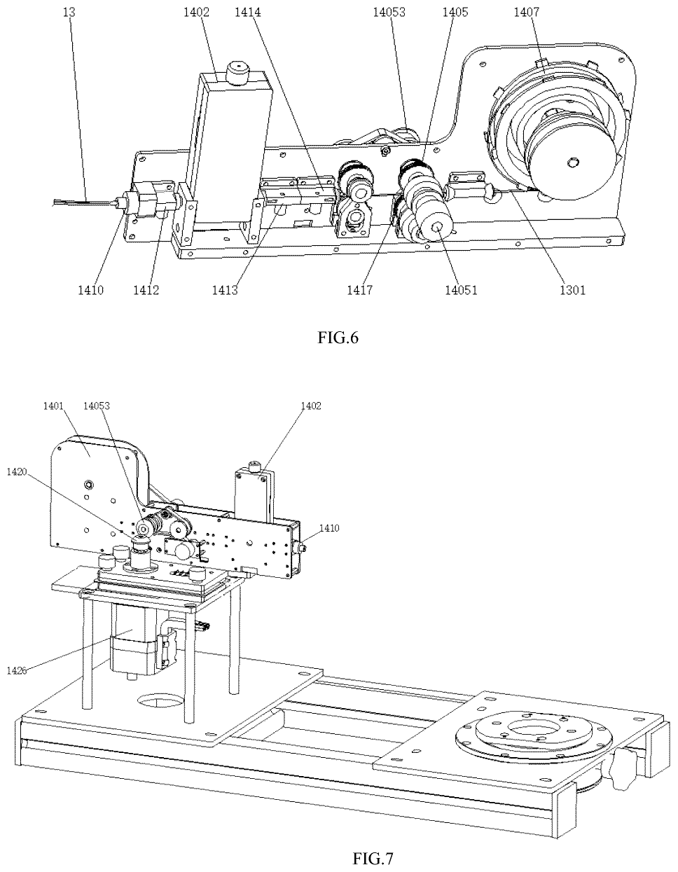

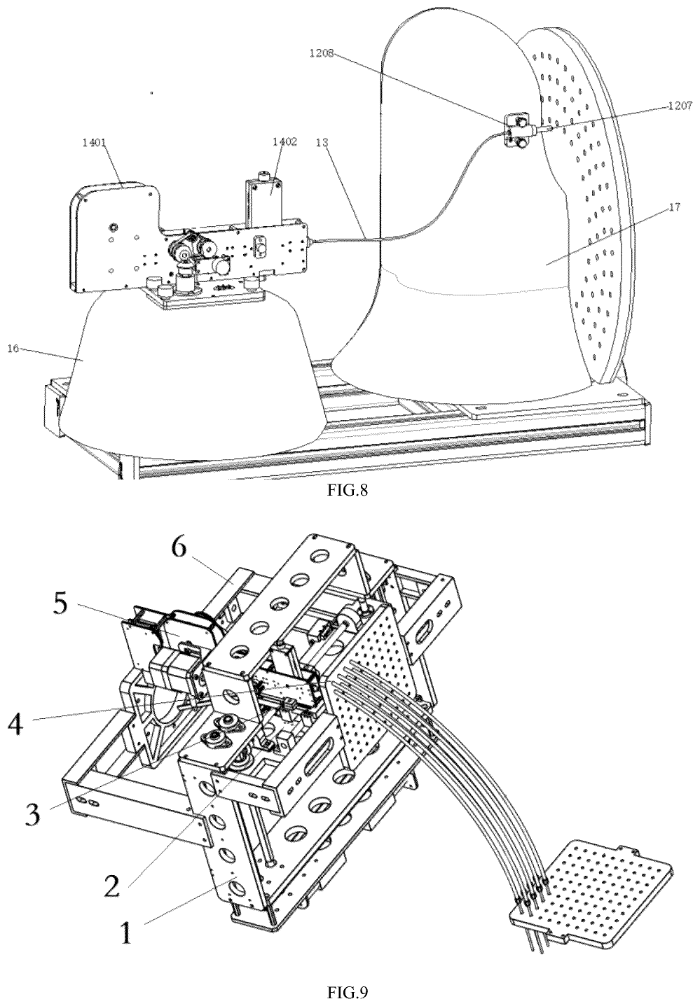

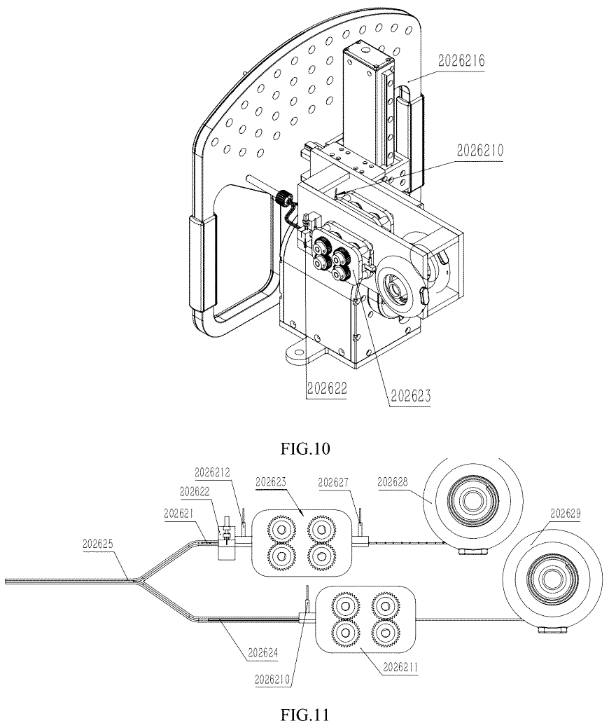

Referring to FIGS. 5-8, the wire driving mechanism 14 comprises a seed applicator body and its driving mechanism. The driving mechanism is used to transfer the torque from the driving motor to the interior mechanism of the seed applicator body, and receive the signal of the travelling switch and encoder inside the seed applicator.

The seed applicator body comprises an applicator body 1401, a radioactive source feeding mechanism 1402, a driving friction wheel, a wheel pressing structure 1417, and a wheel-shaped container 1407 set in the applicator body 1401. The radioactive source feeding mechanism 1402 adopts a seed magazine or a seed strand magazine which detachably installs on the applicator body 1401. Radioisotope seeds are stored in the radioactive source feeding mechanism 1402. These seeds are pushed by a flexible pushing wire 1301. The wheel pressing structure 1417 press the driving friction wheel to clamp the flexible pushing wire 1301 and drive the flexible pushing wire to movement back and forth. The rear part of the flexible pushing wire 1301 is stored by the wheel-shaped container 1407.

In this embodiment, the radioactive source feeding mechanism 1402 is a linear magazine. All seeds or seed strands are arranged sequentially within the linear storage slot of the linear magazine. In other embodiments, the radioactive source feeding mechanism 1402 could also adopts a drum magazine structure.

In this embodiment, the applicator body 1401 is installed with a tube connector 1410. The tube connector 1410 connects to the seed outlet of the radioactive source feeding mechanism 1402. Between the tube connector 1410 and the seed outlet of the radioactive source feeding mechanism 1402, there is a connect channel base A 1412. The connect channel base A 1412 has an output channel that connects the tube connector 1410 with the seed outlet of the radioactive source feeding mechanism 1402. This output channel is designed for the passage of radioisotope seeds and the flexible pushing wire 1301. Outside the entry of the flexible pushing wire 1301 in the radioactive source feeding mechanism 1402, there are connect channel base B 1413 and connect channel base C 1414. Both connect channel base B 1413 and connect channel base C 1414 have a delivery channel designed for the passage of the flexible pushing wire 1301. When the flexible pushing wire 1301 moves to push seeds, its path sequentially passes through connect channel base C 1414, connect channel base B 1413, the radioactive source feeding mechanism 1402, connect channel base A 1412, and a tube connector 1410. The connect channel base C 1414, connect channel base B 1413, the radioactive source feeding mechanism 1402, connect channel base A 1412, and the tube connector 1410 can all be considered as part of the wire output channel 13. The applicator body 1401 is equipped with travelling switches at the locations of the connect channel base A 1412, the connect channel base B 1413, and the connect channel base C 1414, respectively. These switches are used to detect whether the flexible pushing wire 1301 has passed through, thereby determining the accurate position of the flexible pushing wire 1301. The three travelling switches adopt conductive travelling switches. Alternatively, the travelling switch can be a mechanical travelling switch, photoelectric travelling switch, inductive proximity switch, capacitive proximity switch, or magnetic proximity switch.

In this embodiment, the connect channel base A 1412 and the tube connector 1410 can connect to each other or individually secure to the applicator body, or they can be formed as single integral unit. The connect channel base B 1413 and the connect channel base C 1414 can connects to each other or individually secure to the applicator body, or they can be formed as single integral unit.

The driving mechanism installs on the driving motor 1426 of the applicator body 1401. The output axis of the driving motor has an input bevel gear 1420 secured on it. The second friction wheel axis 14051 has a friction wheel bevel gear 14053 installed at its end. The input bevel gear 1420 meshes with the friction wheel bevel gear 14053, thereby transmitting power to the driving friction wheel.

In other embodiments, the main control body is equipped with swivel casters at its base, which makes it convenient for medical workers to move the seed implantation device and arrange it within the operating room. There is an operation panel and functional buttons on the main control body, facilitating the medical workers in operating the seed implantation device.

Due to the positioning errors of the first motion platform, it is often impossible to precisely align the docking tube structure with each flexible delivery tube mounted on the first connection part. So, there is a conical hole on the inlet of each flexible delivery tube or each connection hole on the first connection part. The conical hole can achieve automatic alignment. As long as the deviation is not significant, it can automatically adjust the docking tube structure for alignment. During this process, the docking head 1207 must be “floating” connected to the docking tube structure, which means it has the capability to automatically adjust. The docking tube structure should be able to reset automatically once the docking is separated. The “floating” connection can be established within the docking tube structure. For example, by placing an elastic ring between the docking head 1207 and the docking head connecting base 1208. The elastic ring can deform, thereby automatically accommodating external forces, achieving a floating connection. Alternatively, the back-and-forth motion module main body 1205 can be “floating” connected to the radial motion connection block 12042. The material of the elastic ring can be made of flexible materials with elasticity, such as plastic, rubber, latex, silicone, and other elastomer materials. The elastic ring can also be a spring. The “floating” connection can also be composed of other forms of guiding elements and elastic elements, as detailed in the following text.

In this embodiment, the seed implantation process is as following: the radioactive source feeding mechanism 1402 is a seed magazine or a seed strand magazine. The flexible delivery tube is connected on the connection holes of the first connection part 11. By the rotation and radial movement of the first motion platform 12, the docking head 1207 is aligned with the rear end of the flexible delivery tube or the connecting holds on the first connection part 11 is achieved. When the conical hole is set on the tail of the flexible delivery tube, the connection holes on the first connection part 11 exposes the entire tail of the flexible delivery tube. Then the back-and-forth movement of the first motion platform 12 achieves the docking of the conical surface on the docking head 1207 with the conical hole on the tail of the flexible delivery tube.

The wire driving mechanism 14 drives the flexible pushing wire 1301 to push out seeds or seed strands from the radioactive source feeding mechanism 1402. These seeds or seed strands are then delivered through the wire output channel 13, the docking head 1207, the flexible delivery tube, and the needle trocar and implanted in or around the tumor target area within the human body. The radioactive source feeding mechanism 1402 can also be replaced with a cutting mechanism or a seed strand feeding mechanism for supplying (see below).

### Embodiment 2

As illustrated in FIG. 10 and FIG. 11, the radioactive source feeding mechanism adopts a seed strand for supplying. The radioactive source feeding mechanism comprises the seed strand driving mechanism 202623, the seed strand output channel, and the cutting mechanism 202622, the seed strand storage mechanism 202628. It continuously outputs seed strands 202621 or seed strand casings from the seed strand storage mechanism 202628 by the seed strand driving mechanism 202623 and cuts them to the desired length by the cutting mechanism 202622, thereby achieving the supply of seed strands 202621 or seed strand casings. When the seed strand driving mechanism 202623 outputs a seed strand casing, the radioactive source feeding mechanism also includes a seed embedding mechanism. This seed embedding mechanism is capable of inserting seeds and/or spacers into the seed strand casing from one end or the side, thereby forming a complete seed strand 202621. The seed strand driving mechanism 202623 connects to the seed strand output channel. The seed strand output channel is a rigid structure or a flexible structure that can be bent. The seed strand output channel is connected with a divided channel 202625 or a channel switching mechanism. And the divided channel 202625 or the channel switching mechanism is also connect to the wire output channel.

The wire output channel and the seed strand output channel converge into single channel through the divided channel 202625. The first branch of the divided channel 202625 connects to the wire output channel, and the second branch of the divided channel connects to the seed strand output channel. The main channel of the divided channel connects to the mixed output channel, which docks with the flexible delivery tube. The mixed output channel is a rigid structure or a flexible structure that can be bent.

When implantation is required, the seed strand 202621, which has been cut to the target length, is pushed to the main channel of the divide channel 202625 by the seed strand driving mechanism 202623. The seed strand driving mechanism 202623 retracts the uncut seed strand 2026221 out of the main channel of the divided channel 202625. Subsequently, the second flexible pushing wire 202624, driven by the flexible wire driving mechanism 2026211, moves forward into the main channel of the divided channel 202625, pushes the cut seed strand 202621 forward. The seed strand 202621 is then pushed along the flexible delivery tube and the needle trocar connected at the front end of the flexible delivery tube. The seed strand is pushed into the biological tissue, thus the implantation of the seed strand is completed. Subsequently, the second flexible pushing wire 202624 is retracted into the wire storage mechanism 202629.

The divided channel 202625 can also be a multi-channel divided channel, and the number of branches is greater than 2. It is equipped with a plurality of seed strand driving mechanisms 202623, each designed to drive seed strands 202621 of different types or with different spacer lengths. The output channels of these different seed strand driving mechanisms 202623 connects to the various branches of the divided channel 202625, allowing for the convergence of different types of seed strands, severed to the target length, into the main channel. This facilitates the setting of different types of seed strands 202621 according to the surgical requirements, and the seed strands can then be implanted into the biological tissue by the second pushing wire 202624.

The cutting mechanism 202622 is positioned at each seed strand output channel, each divided channel, or the mixed output channel.

The main channel of the divided channel 202625 sets a one-way valve mechanism designed to prevent the seed strand 202621 from flowing back. This one-way valve mechanism is either a damping block or an elastic valve component.

The cutting mechanism 202622 adopts one or more combination of a guillotine-style cutting mechanism, a scissor-style cutting mechanism, and a circular cutting mechanism. The guillotine-style cutting mechanism accomplishes the cutting by the movement of a single-side blade. The scissor-style cutting mechanism achieves the cutting by making both blades move towards each other simultaneously. The circular cutting mechanism completes the cutting by making at least three blades move towards the center point simultaneously.

Additionally, it incorporates a cutting power source that connects to the cutting mechanism 202622 by the cutting transmission mechanism, or directly connects, thereby transferring the power to the cutting mechanism 202622 to perform the cutting action. The cutting transmission mechanism can be one or more combination of the following: a linkage mechanism, a lead screw and nut mechanism, a gear mechanism, a belt transmission mechanism, or a cam mechanism. The cutting power source can be a combination of one or more of the following: an electric motor, a pneumatic push rod, a pneumatic motor, a hydraulic push rod, or a hydraulic motor.

In the present embodiment, the first motion platform 2026216 operates to dock the docking tube with the rear end of the flexible delivery tube or the connection holes on the first connection part to complete the docking. The seed strand 202621, after being cut, is delivered into the docking tube through the coordinated operation of the seed strand driving mechanism 202623, travelling switch C 2026212, travelling switch D 202627, travelling switch E 2026210, and the cutting mechanism 202622. The second flexible pushing wire 202624, driven by the flexible wire driving mechanism 2026211, moves forward to push the severed seed strand 202621 into the human body, thereby completing the seed strand implantation in one go.

In the present embodiment, the position of the cutting mechanism 202622 can also be placed at the docking tube (that is, after the tubes converge). This enables the seed strand to be driven to the docking tube, next the seed strand is cut, and then retracts the uncut seed strand out of the docking tube structure. Subsequently, the second flexible pushing wire can push the cut seed strand.

The divided channel can be replaced by a channel switching mechanism. Initially, the channel switching mechanism docking the seed strand output channel with the mixed output channel or the flexible delivery tube, and pushing the cut seed strand into the mixed output channel or the flexible delivery tube. Subsequently, docking the wire output channel with the mixed output channel or the flexible delivery tube, and the flexible pushing wire pushes the cut seed strand forward until it is implanted into the biological tissue.

### Embodiment 3

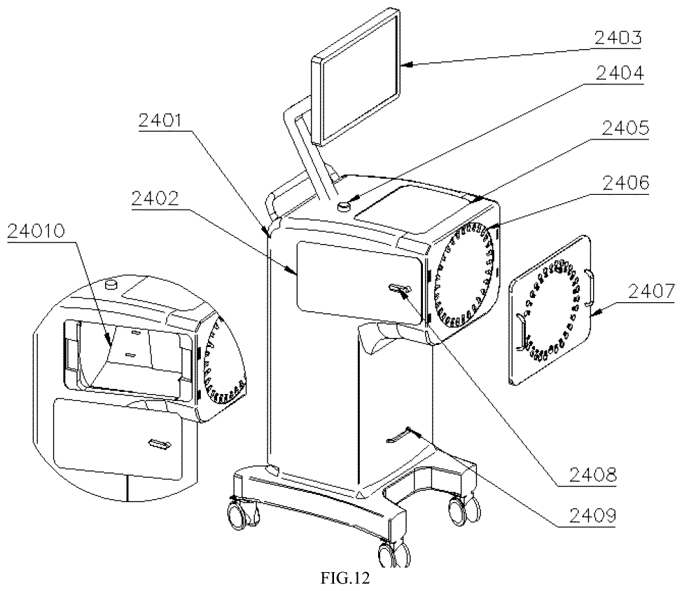

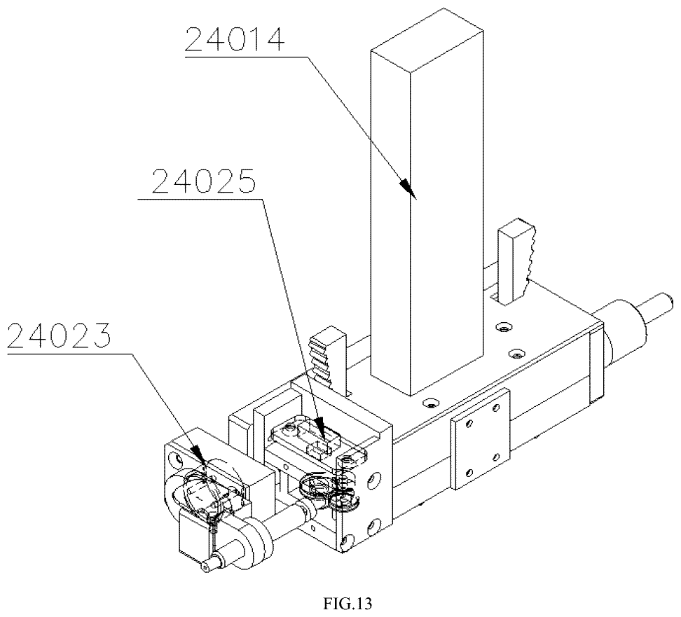

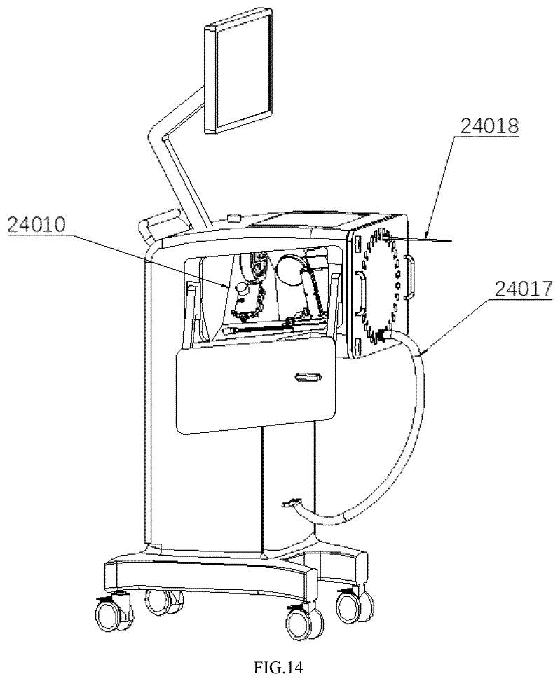

As illustrated in FIGS. 12-22, a multi-channel radioactive source implantation device, in this embodiment, the parts that are same as those in Embodiment 1 are not repeated. The differences are as follows:

The radioactive source feeding mechanism uses a magazine for feeding. The radioactive source feeding mechanism is directly connected to the wire output channel. Seeds, or seed strands, or seed strand casings are loaded into the storage slot or storage holes within the magazine. The feeding mechanism installed on the magazine is used to place seeds, or seed strands, or seed strand casings at the front end of the pushing wire for feeding.

The flexible delivery tube includes an inner tube and an outer tube, the outer tube is sleeved outside the inner tube, the inner tube is clearance-matched with the outer tube and is pluggable relative to the outer tube. The inner tube is configured to be connected with a puncture needle, and the inner tube and the outer tube are made of a plastic tube or a medical braided tube.

The plastic tube is made of a PTFE material.

A first motion platform includes a back-and-forth movement module and a rotational movement module, and the first motion platform realizes the movement of one end of the wire output channel in the space through a one-direction rotational movement and a one-direction linear movement.

When the first motion platform is disposed inside a main body housing and includes the back-and-forth movement module and the rotational movement module, a front side of the main body housing is a first connection portion.

The first connection portion is mounted with a connection part, the connect part is provided with a plurality of connection holes on a rotational track of the first motion platform. Each connection hole may be connected to a flexible delivery tube, and the other end of the flexible delivery tube is connected with a puncture needle.

The motion platform is in a shape of a drum, and a wire driving mechanism is disposed in the drum. The rotational movement module drives the drum to rotate around the axis of the drum, and the back-and-forth movement module is disposed in the drum for driving one end of the wire output channel to move forward and backward.

A door is disposed on a left and/or the right side of the main body housing, and a combination of a wire driving mechanism, a stylet pulling mechanism, a magazine base, and a docking tube structure may be mounted on the first motion platform by opening the door. The stylet pulling mechanism, the magazine base, and the docking tube structure are mounted perpendicular to the first connection portion, and a magazine is mounted on the magazine base or mounted on the wire driving mechanism.

A combination of a wire driving mechanism, a magazine base, and a docking tube structure are mounted on the first motion platform. The magazine base, and the docking tube structure are mounted perpendicular to the first connection portion, and a magazine is mounted on the magazine base or mounted on the wire driving mechanism.

When the wire driving mechanism and the magazine base are installed on the first motion platform, the wire driving mechanism and the magazine base are connected by a flexible connection tube, and at this time, the flexible connection tube is a part of the wire output channel, and an outlet of the magazine base is a docking head, which is a terminal end of the wire output channel.

When the wire driving mechanism and the docking tube structure are mounted on the first motion platform, the wire driving mechanism and the docking tube structure are connected by the flexible connection tube, and at this time, the flexible connection tube is a part of the wire output channel, an outlet of the docking tube structure is a terminal end of the wire output channel, and the docking tube structure adopts a floating docking tube structure which can float within a certain range.

The flexible delivery tube is quickly mounted to the connection hole of the first connection part by means of a quick connector, and the first connection part is provided with a synchronized connector locking mechanism, which may lock the quick connectors of all flexible delivery tubes simultaneously.

As shown in FIG. 21, the synchronized connector locking mechanism includes a connector clamping plate 24038 disposed within the first connection part, and connector clamping portions are arranged in a circular array on the connector clamping plate 24038. In an unlocked state, all connector clamping portions are staggered with the connection holes, and the quick connector 24029 of the flexible delivery tube can be inserted into the connection holes. When locking, the connector clamping plate 24038 is driven by a clamping driving mechanism to rotate around a center axis, and the connector clamping portions extend into the connection holes. A side of the quick connector 24029 of the flexible delivery tube is provided with a slot, and the quick connector 24029 of the flexible delivery tube is fixed on the first connection part when the connector clamping plate 24038 is rotary to clamp into the slot.

The clamping driving mechanism is electrically or manually actuated and drives the connector clamping plate to rotate around the central axis through cams, gears, or belt drives.

A stylet is disposed inside the flexible delivery tube, extends all the way along the flexible delivery tube, and fills up a space inside the puncture needle connected at a front end of the flexible delivery tube, so as to avoid the blood from pouring into the puncture needle to coagulate and form a clogging. The first connection part is also provided with n synchronized stylet locking mechanism, which may lock the stylets inside all flexible delivery tubes simultaneously.

The synchronized stylet locking mechanism includes a stylet clamping plate 24040 disposed inside the first connection part, stylet clamping portions are arranged in a circular array on the stylet clamping plate 24040. In the unlocked state, all stylet clamping portions are staggered from the connection holes, and the quick connector 24029 of the flexible delivery tube can be inserted into the connection holes. When locking, the stylet clamping plate 24040 rotates around a central axis driven by a stylet driving mechanism, and each of the stylet clamping portions inserts into the interior of each of the connection holes. An opening connected to an elastic body 24041 inside the quick connector 24029 is provided on the side of the quick connector 24029 of the flexible delivery tube, and the elastic body 24041 adopts one or a combination of an elastomer, a rubber tube, or a TPU tube. The elastic body 24041 is adjacent to the stylet, and the stylet clamping plate 24040 presses against the elastic body 24041 during rotation to press down the stylet, thereby realizing the locking.

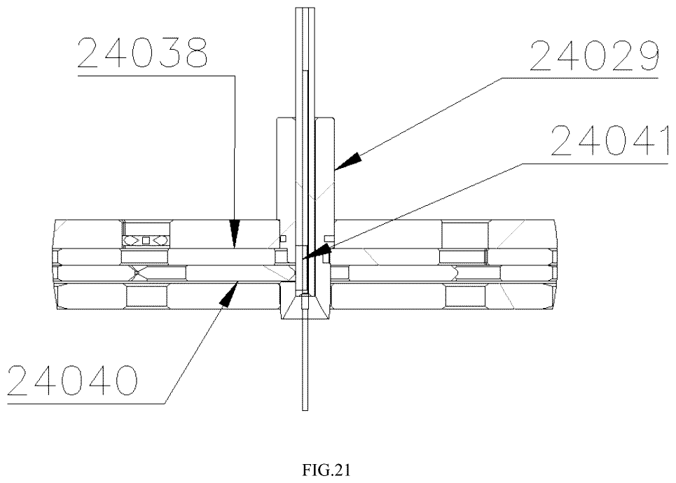

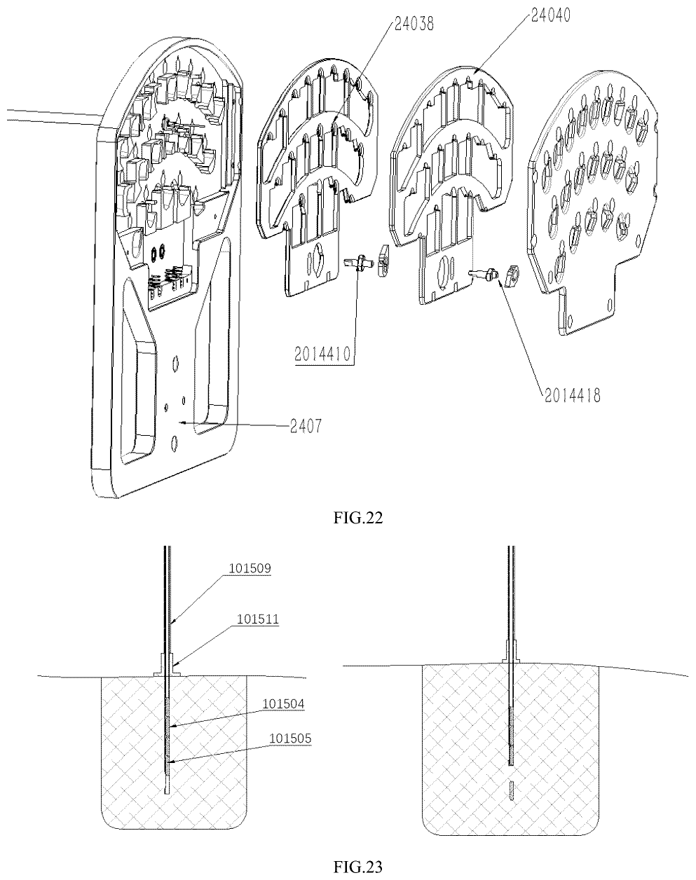

Or, as illustrated in FIG. 22, during the locking process, the clamping actuation mechanism 2014410 drives the connector clamping plate 24038 to translate, misaligning the clamping portions with the connection holes. The side of the quick connector of the flexible delivery tube has a slot. The clamping portions of the connector clamping plate 24038 is engaged into the slot through its translation, thereby securing the quick connector of the flexible delivery tube to the docking plate 2407. When locking the flexible stylet, the stylet clamping driving mechanism 2014418 drives the stylet clamping plate 24040 to translate, misaligning the stylet clamping portions with the connection holes. The side of the quick connector of the flexible delivery tube has an opening that is connected to the internal elastic portion, with the inside of the elastic portion adjacent to the flexible stylet. As the stylet clamping plate 24040 translates, it presses against the elastic portion, so as to compress the flexible stylet through the elastic portion, achieving the flexible stylet locking.

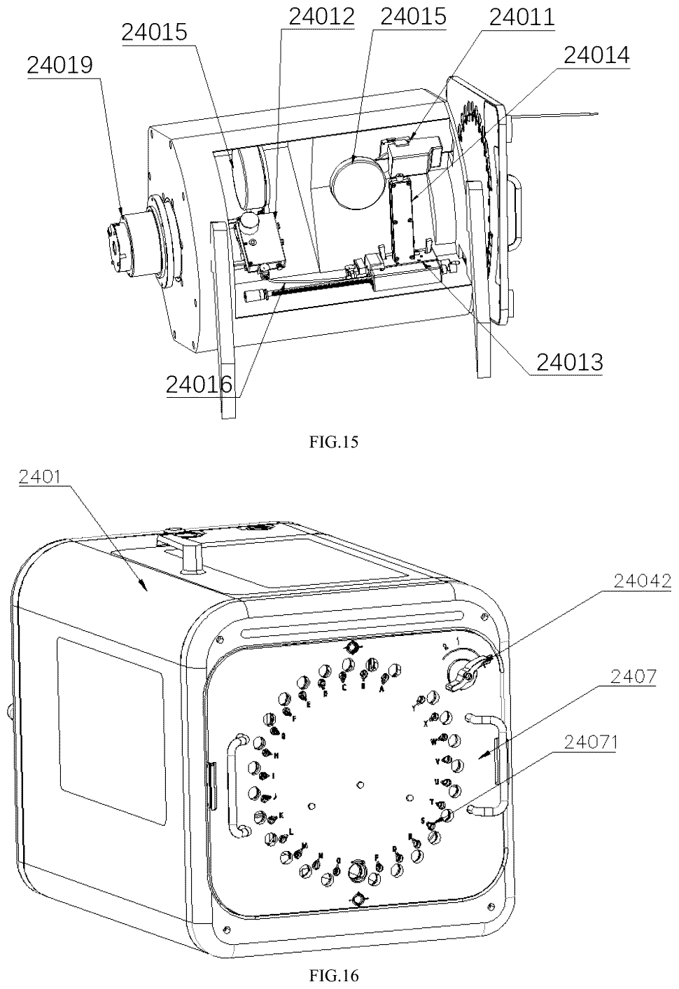

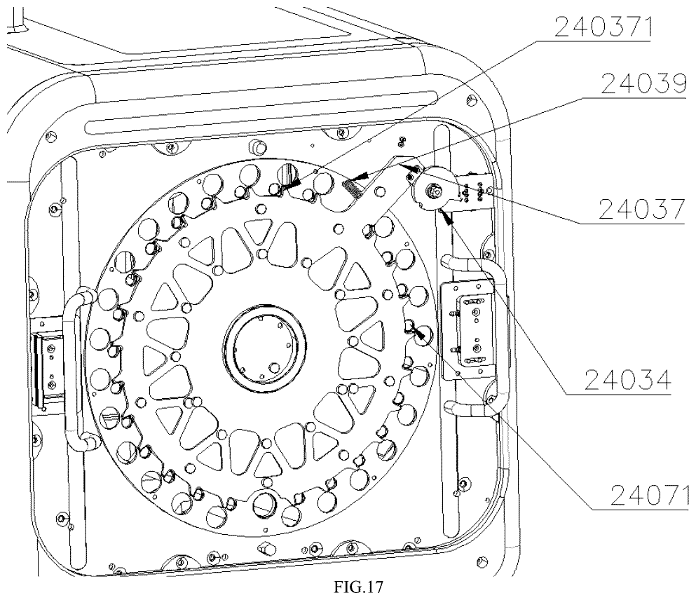

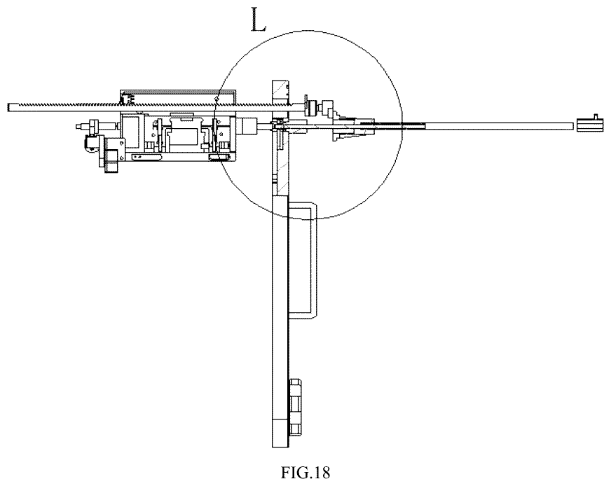

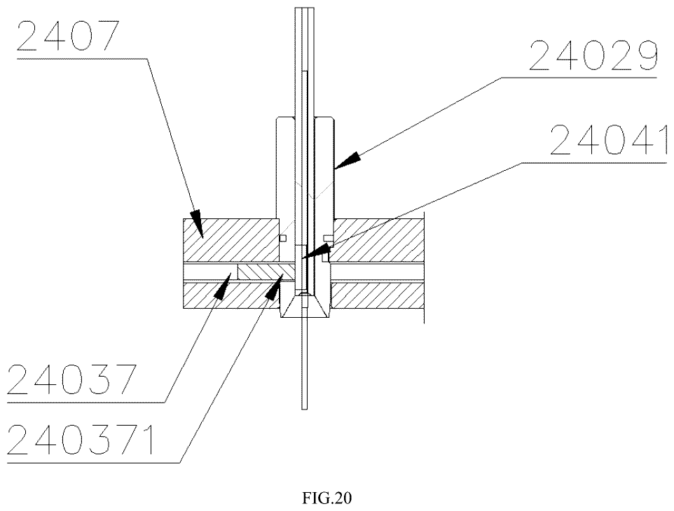

Or, as illustrated in FIG. 16, FIG. 17 and FIG. 20, the synchronized connector locking mechanism is the synchronized stylet locking mechanism, the connector clamping plate is the stylet clamping plate, and the clamping driving mechanism is the stylet driving mechanism, and when the clamping driving mechanism drives the connector clamping plate to rotate around a center axis by a first angle, the connector clamping plate inserts into the slot of the quick connector of the flexible delivery tube while not holding against the elastic body. The connector clamping plate fixes the quick connector of the flexible delivery tube on the first connection part, which just realizes locking of the quick connector of each flexible delivery tube but not realizes locking of the style inside each flexible delivery tube. When the clamping driving mechanism drives the connector clamping plate to rotate around the center axis by a second angle, the connector clamping plate presses against the elastic body during rotation, and then presses the stylet, which realizes locking of the quick connector and the style inside each flexible delivery tube.

The stylet needs to be pulled out before an implantation, the stylet is a flexible wire with elasticity, which may be bent under an external force and restored to a straight state after the external force is withdrawn. A length of the stylet is greater than 600 mm. A rear end of the stylet extends backwardly by a portion from a rear end of the flexible delivery tube, and the rear end of the stylet is provided with a first stopping step, and when the first stopping step is pressed against the rear end of the flexible delivery tube, a distance between a front end of the stylet and the front end of the puncture needle connected to the front end of the flexible delivery tube is no more than 5 mm.

As shown in FIG. 12 to FIG. 23, the first motion platform is the drum, the drum is disposed inside the main body housing, the drum has a one-direction rotational movement and a one-direction linear movement. The wire driving mechanism, the stylet pulling mechanism, and the magazine base are provided on the drum. The radioactive source feeding mechanism adopts a magazine, the first connection part adopts a docking plate, and a needle pulling driver drives the inner tube or outer tube to make a relative sliding movement between the inner tube or outer tube by direct pushing and pulling.

A main body of a radioactive source implantation mechanism includes a main body housing 2401, a door 2402, a display screen 2403, an emergency stop button 2404, an observation window 2405, a docking plate panel 2406, a docking plate 2407, a door handle 2408, a support column 2409, and a drum 24010.

Before the surgery, the door 2402 is in a closed state, the door 2402 is opened by rotate the door handle 2408, the door 2402 is located on the left and/or right side of the main body housing 2401, accessories are installed inside the drum 24010. Specifically, the stylet pulling mechanism 24011, the wire driving mechanism 24012, and the magazine base 24013 are installed in corresponding positions in the drum 24010. The stylet pulling mechanism 24011 and the magazine base 24013 are vertically mounted on the docking plate panel 2406 (referring to FIG. 15). A stylet storage wheel 24015 is disposed at the rear side of the stylet pulling mechanism 24011, and a wire storage wheel 24020 is provided at the rear side of the wire driving mechanism 24012, and the flexible pushing wire 24080 is stored inside the wire storage wheel 24020, and a main material of the flexible pushing wire 24080 is a Ni—Ti alloy wire with a certain degree of toughness. The front end of the Ni—Ti alloy wire is a segment of flexible plastic material. At the position of the flexible segment, the diameter of the Ni—Ti alloy wire is reduced, and the exterior is filled by the flexible segment, keeping the overall outer diameter of the flexible pushing wire consistent. The flexibility of the flexible pushing wire at the position of the flexible segment and its front end is better. When the flexible pushing wire moves inside the connection tube 24016 and the flexible delivery tube 24018 and encounters a bending position, it has better self-adaptability, which makes it less likely to scratch the flexible delivery tube 24018. At the same time, it can reduce the friction between the flexible pushing wire and the inner wall of the flexible delivery tube 24018. One end of the connection tube 24016 is connected to the wire driving mechanism 24012, the other end of the connection tube 24016 is connected to a force transducer 24023 on the magazine base 24013, and there is a bending section of the connection tube 24016. A magazine 24014 is fitted into the magazine base 24013. The docking plate 2407 is installed on the docking plate panel 2406 (first connection portion). A stylet recycling tube 24017 is fixed on a stylet disposal hole of the docking plate 2407. The door 2402 is closed when the installation is complete.

At the beginning of the surgery, a corresponding count of flexible delivery tubes 24018 are prepared in accordance with the count of puncture needles required for the surgery, and quick connectors 24029 of a plurality of flexible delivery tubes 24018 are sequentially installed into connection holes 24071 on the docking plate 2407, a locking handle 24042 is sequentially rotated to a first gear position, a cam 24034 internally connected with the locking handle 24042 rotates to rotate a connector locking plate 24037, the connector locking plate 24037 is provided with a plurality of convex plates 240371 corresponding to the connection holes 24071, and the convex plates 240371 rotate to be embedded in the slot on a side of quick connectors 24029, but not hold against the elastic body 24041, thereby fixing the quick connectors 24029 of the flexible delivery tube 24018 to the docking plate 2407. Then the locking handle 24042 is rotated to a second gear position, the cam 24034 internally connected with the locking handle 24042 rotates to cause the connector locking plate 24037 to continue to rotate, and the convex plate 240371 rotates to press against the elastic body 24041 inside the quick connectors 24429, and the elastic body 24041 simultaneously presses against a stylet inside the flexible delivery tube 24018. After locking the connector locking plate 24037, the mechanism simultaneously locks all the quick connectors 24429 of the flexible delivery tube 24018 and the stylets inside the delivery tube on the docking plate 2407.

On the other side, an operator holds a puncture needle handle and pierces the puncture needle 18 into a target surgical position of a target object 1002 during the surgery. After the puncture is completed, a needle stylet inside the puncture needle (the needle stylet is very short in length and is used only for puncture) is pulled out, and then the long flexible stylet protruding from the front end of the flexible delivery tube is inserted into the puncture needle to fill the space inside the puncture needle, and then the rear end of the puncture needle is connected to a second quick connector at the front end of the flexible delivery tube, forming a complete implantation channel.

Turning the locking handle 24042 along an opposite direction to the first gear position, a spring 24039 connected with the connector locking plate 24037 springs back to cause the convex plate 240371 to loosen the elastic body 24041 and the stylet, but still lock the quick connectors 24029, and the above steps may be repeated until all flexible delivery tubes 24018 are properly installed and the puncture needles have all punctured to the target positions.

When a user starts the seed implantation system, the machine starts to operate, a motor 24019 controls an internal drum 24010 to rotate, thereby making the stylet pulling mechanism 24011 move to align with a flexible delivery tube 24018. Then the stylet pulling mechanism 24011 starts to work and at the same time moves forward to pull out the stylet (by docking with the tail portion of the stylet) inside the flexible delivery tube 24018, and the stylet may be delivered to the stylet storage wheel 24015 (a concave structure) on the rear side of the stylet pulling mechanism 24011, the stylet automatically coil inside the stylet storage wheel 24015 under its own elastic action, and drive the stylet storage wheel 24015 to rotate synchronously. After the stylet is completely pulled out of the flexible delivery tube 24018, the stylet pulling mechanism 24011 returns backwardly. The stylet pulling mechanism 24011 is controlled to move back and forth through a linear movement mechanism, and the linear movement mechanism is one or a combination of a screw nut mechanism, an electric pushing rod, and a rack and pinion mechanism. The motor 24019 controls the drum 24010 to rotate so that the stylet pulling mechanism 24011 is aligned with the recycling tube 24017 (aligned with the recycling hole of the docking plate 2407), and the stylet pulling mechanism 24011 then works to completely spit out the stylet and inserts the stylet into the recycling tube 24017.

Subsequently, the motor 24019 drives the drum 24010 to rotate so that the front end of the wire output channel is aligned to the flexible delivery tube 24018 in which the stylet has just been pulled, and the front end of the wire output channel is docked to the quick connector 24029 of this flexible delivery tube 24018. The front end of the wire output channel is controlled to move back and forth through the implantation docking mechanism, and then the wire driving mechanism 24012 pushes out the flexible pushing wire inside the wire storage wheel 24020, and the flexible pushing wire pushes out a seed inside the magazine 24014 to the docking tube structure 24026 at the front end of the wire output channel, and repeats until the wire output channel is piled up with a specified number of seeds, and then all of the seeds in front of the flexible pushing wire at once are pushed into the flexible delivery tube 24018 until they are delivered into a second puncture needle 101504.

In this scheme, the magazine base 24013 is disposed at the front portion of the wire output channel, a passive measurement wheel is provided inside the magazine base 24013 to press the flexible pushing wire, the friction generated during the movement of the flexible pushing wire drives the passive measurement wheel to rotate, and the rotation angle of the passive measurement wheel is measured by a encoder 24025. A displacement of the flexible pushing wire may be converted by the encoder 24025. Since an encoder module and a measuring wheel are also disposed inside the wire driving mechanism 24012, then the readings of the two encoders may be used to determine whether the passive measurement wheel is slipping, so as to ensure the reliability of a measurement result based on two measurement results, and through reciprocal movements, seeds being pushed reach a specified count. Then the wire driving mechanism 24012 drives the flexible pushing wire to push out the seeds through the flexible delivery tube 24018 to the front end of the puncture needle. In this process, if a resistance subjected by the flexible pushing wire becomes larger during movement, its reaction force may be transmitted to the force transducer 24023 connected with the connection tube 24016 and fed back.

In another embodiment, reflective stripes or color stripes are provided on the flexible pushing wire, and a photoelectric sensor is disposed in the magazine base 24013 or the wire driving mechanism 24012, so that when the relative displacement of the flexible pushing wire and the photoelectric sensor occurs, the reflective stripes or color stripes may synchronously generate a pulse signal, and an actual displacement of the flexible pushing wire may be measured by sampling the pulse signal, and this solution can avoid slippage between the passive measurement wheel and the flexible pushing wire that may affect the measurement accuracy and reliability of the encoder.

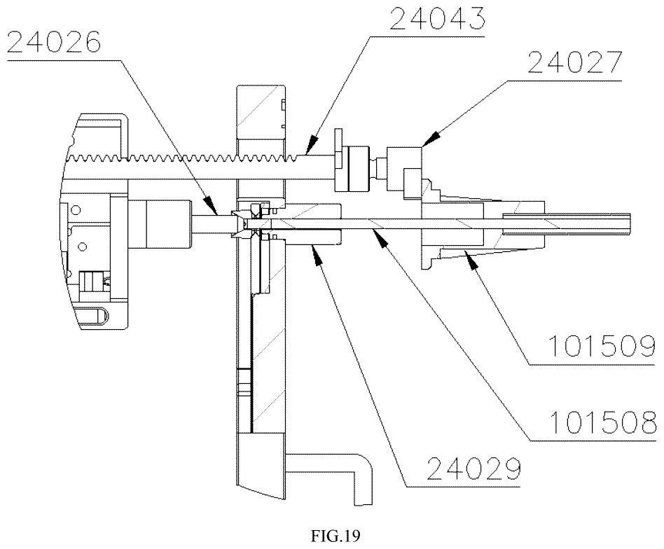

Subsequently, a needle pulling rod 24043 is pushed forward until it comes into contact with an outer tube base 101509 of outer tube, at which time a contact signal is sensed by a contact sensor 24027, and the contact sensor 24027 is one or a combination of a force transducer, a mechanical switch, an inductive switch, and a photoelectric switch. Then, as shown in FIG. 19 and FIG. 23, when a plurality of seeds 101505 reach a position of the second puncture needle 101504 connected with the flexible delivery tube, the wire driving mechanism 24012 may push out the first seed into the human body, and then the needle pulling rod 24043 may continue to push the outer tube base 101509 to move forward, and the other end of the outer tube may be pressed against a surface of the body or a puncture template (which may also be an instrument such as a prostate puncture template, etc.), and when the outer tube base 101509 is pushed forward, an inner tube 101508 moves relative to the outer tube and pulls the second puncture needle 101504 connected at the front end of the inner tube partly out of the human body, and then the wire driving mechanism 24012 may continue to push out a next seed 101505 until the plurality of seeds are implanted respectively through repeated operations.

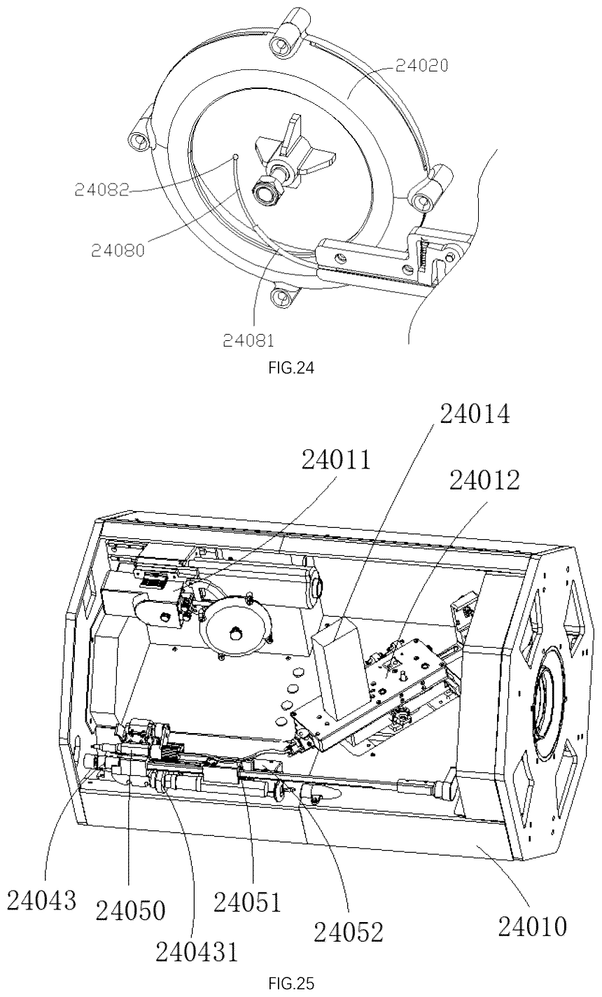

The wire storage wheel 24020 is a wheel-shape container with a concave inner surface (referring to FIG. 24), the wheel-shape container may rotate freely around an axis thereof, and an edge of the wheel-shape container is a concave structure, a storage region is formed at the concave inner surface of the wheel-shape container. At least one side of the wheel-shape container is provided with an opening, and the flexible pushing wire 24012 is guided by a storage guiding tube 24081 to extend into the wheel-shape container through the opening on the side of the wheel-shape container. When the wire driving mechanism 24012 drives the flexible pushing wire to move back and forth, under an elastic action of the flexible pushing wire, the flexible pushing wire is automatically coiled inside a concave inner surface of the wire storage wheel 24020, and the wire storage wheel 24020 rotates along with the back-and-forth movement of the flexible pushing wire, thereby realizing automatic storage.

In other embodiments, a second stopping step 24082 may be added to the rear end of the flexible pushing wire, and a position of the second stopping step is set to satisfy following principles: when the front end of the flexible pushing wire extends from the flexible delivery tube into the puncture needle and probes out from the front end of the puncture needle by more than a certain length (the length is less than 10 mm), the second stopping step is stuck on the rear step of the storage guiding tube, so as to realize the mechanical position-limiting, which prevents the flexible pushing rod from penetrating further into body tissue, thus ensuring overall safety. Preferably, the second stopping step is an end surface of a stopping tube socketed to the rear end of the flexible pushing wire, or the second stopping step is a welded ball at the rear end of the flexible pushing wire. When the wire driving mechanism 24012 drives the flexible pushing wire to move backward again, the flexible pushing wire is automatically coiled inside the inner concave surface of the wire storage wheel 24020 under the elastic action of the flexible pushing wire, and the wire storage wheel 24020 rotates along with the back and forth movement of the flexible pushing wire, thereby realizing automatic storage.

In other embodiments, a wire connection portion may also be provided on the wire storage wheel 24020, the wire connection portion may be configured to connect and fix the rear end or a middle portion of the flexible pushing wire with the wire storage wheel 24020. A position of the wire connection portion may be set to satisfy following principles: when the front end of the flexible pushing wire extends from the flexible delivery tube into the puncture needle and probes out from the front end of the puncture needle by more than a certain length, the flexible pushing wire is pulled by the wire connection portion so that it is unable to continue to move forward, thereby realizing a mechanical position limiting, which avoids the flexible pushing wire from piercing into the body tissues to cause injuries, and ensuring the overall safety. The wire connection portion adopts a form of threaded connection, bonding, welding, snap connection, and other forms to realize connecting and fixing.

### Embodiment 4

The basic principle of the device in the present embodiment is consistent with that in the embodiment of Embodiment 3. The difference is that the seed feeding mechanism (magazine) is located at the front end of the wire output channel in the embodiment of Embodiment 3, whereas in this embodiment, the seed feeding mechanism (magazine) is located inside the wire driving mechanism.

The first motion platform is installed with a wire driving mechanism and a docking tube structure. The wire driving mechanism and the docking tube structure are connected through a flexible connection tube (forming the wire output channel). The first motion platform is used to achieve relative motion in space of the docking tube structure (equivalent to one end of the wire output channel) and the first connection part, allowing the docking tube structure to dock with any connection hole on the first connection part or the flexible delivery tube. The docking tube structure includes a docking head and a first floating connecting mechanism. The docking head is mounted on the first motion platform via the first floating connecting mechanism which enables the docking head to float within a range.

The first floating connecting mechanism adopts two rotational joint modules.

Alternatively, the first floating connecting mechanism adopts one rotational joint module and one translational module.

Alternatively, the first floating connecting mechanism adopts two translational modules, with the movement paths of the two translational modules being orthogonal to each other or at a certain angle.

Alternatively, the first floating connecting mechanism adopts a supporting base with an elastic ball hinge. The elastic ball hinge consists of a third elastic element and a hinge ball. The docking head is installed at the hinge ball which allows the docking head to pivot through a certain angle. The third elastic element is used to provide elastic resetting after the docking head has been deflected.

When the first floating connecting mechanism adopts two rotational joint modules, the first floating connecting mechanism includes a first link, a second link, and a link seat. The link seat is mounted on the first motion platform. The second link is rotatably set on the link seat through a first rotating shaft and achieves the rotational resetting via the first elastic element. The first link is rotatably set on the second link through a second rotating shaft and achieves the rotational resetting via the second elastic element. The docking head is set on the first link, or the docking head connects to the first link through a quick assembly and disassembly structure.

The first floating connecting mechanism also includes a limiting structure that restricts the rotation angle of the first link and/or the second link.

The second elastic element is the first elastic element. One end of the first elastic element connects to the link seat, and the other end connects to the first link. While the first elastic element facilitates the rotational resetting of the first link, it also enables the rotational resetting of the second link.

As illustrated in FIG. 25, the drum component includes: a drum 24010, a stylet pulling mechanism 24011, a flexible wire driving mechanism 24012, a docking tube structure 24050. The stylet pulling mechanism 24011, the flexible wire driving mechanism 24012, and the docking tube structure 24050 install at corresponding positions inside the drum 24010. The stylet pulling mechanism 24011 and the docking tube structure 24050 are installed inside the drum 24010. And the installation direction is perpendicular to the front face of the drum 24010. The stylet pulling mechanism 24011 is capable of pulling out the flexible stylet from the flexible delivery tube. The magazine 24014 installs on the wire driving mechanism 24012. The flexible wire driving mechanism 24012 connects to the docking tube structure 24050 through a flexible connection tube 24051. The docking tube structure 24050 is installed inside the drum 24010 by an implantation docking module 24052, enabling the docking tube structure 24050 to move back and forth and to dock with different flexible delivery tubes on the first connection parts. After docking is completed, the flexible wire driving mechanism 24012 drives the flexible pushing wire through the radioactive source feeding mechanism, thereby pushing the radioactive source located at the front end of the flexible pushing wire, through the docking tube structure 24050, into the flexible delivery tube, and implanting it into the target body via the needle trocar connected to the end of the external delivery tube.

There is a needle pulling rod 24043 below the docking tube structure 24050. Below the needle pulling rod 24043, there is a worm 240431 that is meshed with it. The worm 240431 connects to the output shaft of the stylet pulling motor.

In the present embodiment, the seed implantation process is as follows: the stylet pulling mechanism 24011 is driven by the rotation of the drum 24010 selectively to align with a flexible delivery tube on the docking plate. The linear motion mechanism drives the stylet pulling mechanism 24011 to approach the flexible stylet inside the flexible delivery tube. The stylet pulling mechanism 24011 is activated to pull out the flexible stylet from the flexible delivery tube. Then, the linear motion mechanism drives the stylet pulling mechanism 24011 to retreat. The drum 24010 continues to rotate, driving the docking tube structure 24050 to selectively align with this flexible delivery tube from which the flexible stylet is pulled out. The implantation docking module 24052 drives the docking tube structure 24050 to move forward and dock with the flexible delivery tube. Then, the flexible wire driving mechanism 24012 drives the pushing wire to move, thereby pushing the radioactive source from the magazine 24014 along the flexible connection tube 24051, through the docking tube structure 24050, the flexible delivery tube, and the needle trocar into the target body.

The docking tube structure 24050 includes a docking head 2405001 and a floating connecting mechanism. The docking head 2405001 is mounted on the implantation docking module 24052 via the floating connecting mechanism which drives the docking head to float within a range that is parallel to the docking plane.

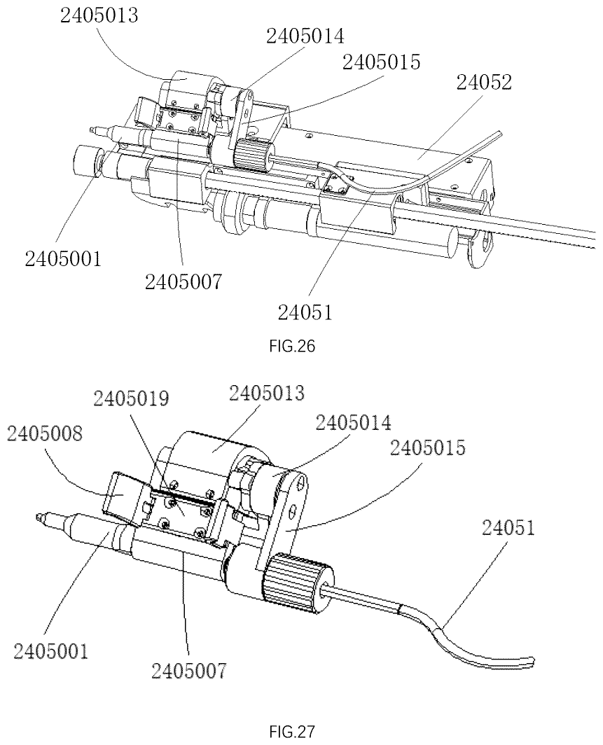

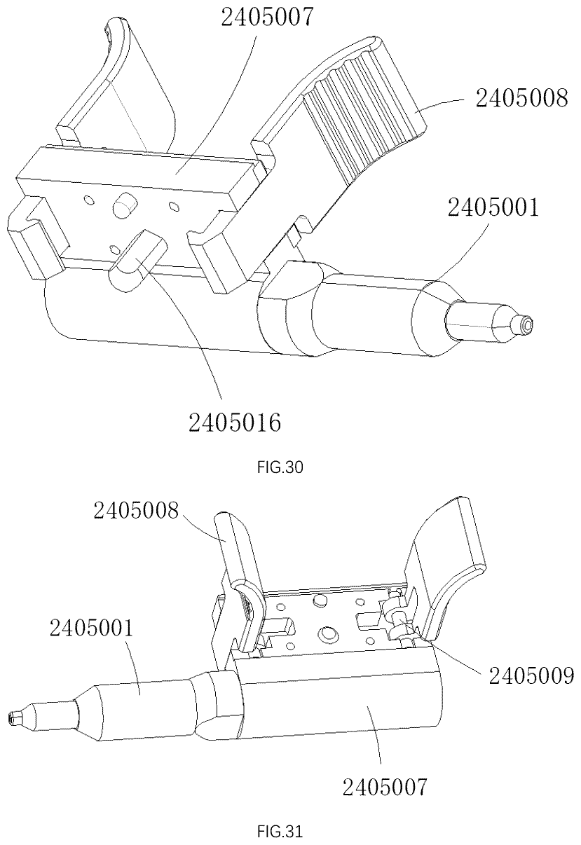

Scheme 1, as illustrated in FIGS. 26-31: the floating connection mechanism employs two rotary joint modules, the floating connection mechanism includes a first link 2405002, a second link 2405003, and a link seat 2405004, the link seat 2405004 is mounted on the implantation docking module 24052, the second link 2405003 is rotationally set on the link seat 2405004 via a first rotary shaft 2405005, and a rotational resetting of the second link 2405003 is realized via a first elastic element, the first link 2405002 is rotationally set on the second link 2405003 via a second rotary shaft 2405006, and the first link 2405002 is rotationally set on the second link 2405003 via the second rotary shaft 2405006, and realizes the rotational resetting of the first link 2405002 through the second elastic element, the docking head 2405001 is connected to the first link 2405002 through a quick-lock structure, and the first elastic element and the second elastic element may adopt a torsion spring structure, respectively, and two torsion springs are mounted at the first rotary shaft 2405005 and the second rotary shaft 2405006, respectively.

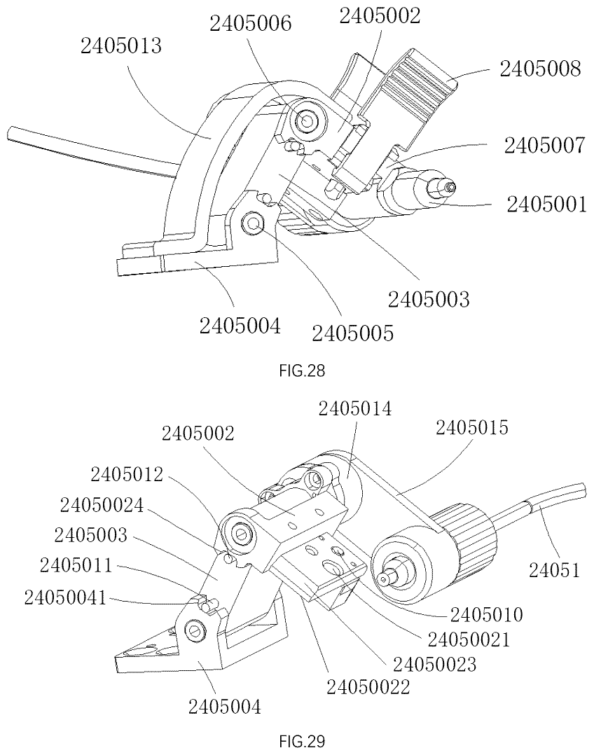

Or, as illustrated in FIG. 28, the second elastic element is the first elastic element, the first elastic element and the second elastic element are a same elastic plate 2405013, the elastic plate 2405013 having a special shape, one end of the elastic plate 2405013 is fixed to the link seat 2405004 and the other end is fixed to the first link 2405002. When the implantation docking module 24052 drive the docking tube structure to move forward for docking, and the docking head 2405001 is not precisely aligned with the flexible delivery tube on the external connection part, the docking head 2405001 contacts with a conical surface at the connection holes or the rear end of the flexible delivery tube on the external connection part, the first link 2405002 and the second link 2405003 may rotate in a small range so as to realize the adaptive alignment docking. When the implantation docking module 24052 drives the docking tube structure to move backwardly, the first link 2405002 and the second link 2405003 are automatically reset under an action of the first elastic element and the second elastic element so as to be prepared for the next docking.

The quick disassembly structure includes a quick disassembly base 2405007, a latch plate 2405008, a third elastic element, and a hinge 2405009. The first link 2405002 has a positioning hole 24050021. The quick disassembly base 2405007 has a positioning pin 2405016 that is shape-adapted to the positioning hole 24050021, serves as the function of positioning and guidance. The docking head 2405001 secures on the quick disassembly base 2405007. The latch plate 2405008 is set on the quick disassembly base 2405007 via the rotation of the hinge 2405009. The third elastic element is a torsion spring, which is fitted over the hinge 2405009, with one end against the quick disassembly base 2405007 and the other end against the latch plate 2405008. The first link 2405002 has a latch block 24050022 for the hook part of the latch plate 2405008 to engage with. The first link 2405002 also has an inclined surface 24050023 near the latch block 24050022, which allows the hook part of the latch plate 2405008 to also open outward under the effect of the inclined plane 24050023. When a quick assembly is needed, aligning the positioning hole 24050021 with the positioning hole 24050021, pressing the latch plate 2405008 to make its hook part opens outward. When the end face of the quick disassembly base 2405007 is in contact with the end face of the first link 2405002, releasing the latch plate 2405008, and under the action of the third elastic element, the hook part of the latch plate 2405008 turns inward, and the hook part of the latch plate 2405008 hooks with the latch block 24050022 for quick locking.

Among, the first link 2405002 is equipped with a detecting switch 2405010. Once the quick disassembly base 2405007 is properly installed on the first link 2405002, which can trigger the detecting switch 2405010, and send a signal to an device controller indicating that the installation is complete. The detecting switch 2405010 can be a micro-switch. Once the quick disassembly base 2405007 is installed in place, its end face comes into contact with the actuator of the micro-switch, thereby triggering the micro-switch.

It also includes a limiting structure that restricts the rotation angle of the first link and/or the second link. The limiting structure adopts a combination of a limiting rod and a limiting slot. When the limiting rod moves to a position where it comes into contact with the two ends of the limiting slot, it serves to restrict the movement, fulfilling its function as a limiting mechanism. Specifically, the second link 2405003 has a first limiting rod 2405011 and a second limiting rod 2405012 at its two ends, respectively. The link seat 2405004 has a first limiting slot 24050041 that matches up with the first limiting rod 2405011, and the first link 2405002 has a second limiting slot 24050024 that matches up with the second limiting rod 2405012. The first limiting rod 2405011 and the first limiting slot 24050041 work together to restrict the rotation angle of the second link 2405003, while the second limiting rod 2405012 and the second limiting slot 24050024 work together to restrict the rotation angle of the first link 2405002.

The first link 2405002 is equipped with a force transducer 2405014. The force transducer 2405014 connects to the end of the flexible connection tube 24051 through a connecting base 2405015, aligning the front end of the flexible connection tube 24051 with the entrance of the docking head 2405001. When the flexible pushing wire encounters resistance, it will generate a corresponding reaction force on the flexible connection tube 24051. This reaction force will be transmitted to the force transducer 2405014 by the connecting base 2405015, and thus be detected by the force transducer. The position of this force transducer can also be set at the connection between the flexible connection tube 24051 and the flexible wire driving mechanism 24012.

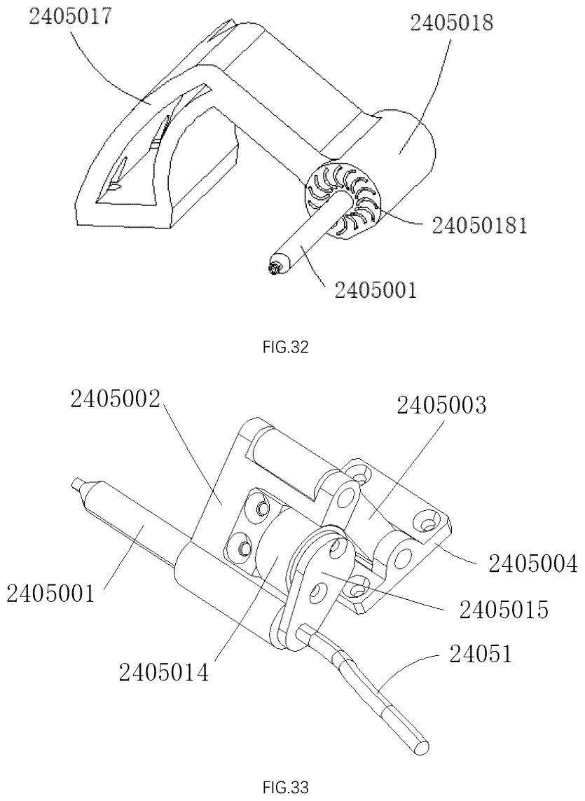

Scheme 2, as illustrated in FIG. 32, the parts of this embodiment that have the same structure as those of Embodiment 1 are not described in detail, and differences are as follows. The floating connection mechanism adopts a support seat with an elastic head portion, it includes a support seat 2405017, the support seat 2405017 being mounted on the implantation docking module 24052, the docking head 2405001 is mounted through the elastic floating ring 2405018, the elastic floating ring 2405018 has a deformation slot 24050181 around a periphery of the docking head 2405001, and the deformation slot 24050181 deforms when subjected to a force. By providing the deformation slot 24050181, the docking head 2405001 performs a certain deformation displacement to realize automatic alignment when the implantation docking module 24052 moves forward for docking, which allows a certain alignment error. The deformation slot 24050181 will resume deformation when the implantation docking module 24052 drives the docking tube structure to move backwardly for resetting the docking head 2405001, thereby preparing for a next docking.

Scheme 3, as illustrated in FIG. 33, the parts of this embodiment that have the same structure as those of Embodiment 1 are not described in detail, and differences are as follows. The docking head 2405001 is fixedly mounted directly to the first link 2405002.

### Embodiment 5

The use method of the multi-channel radioactive source implantation device includes the following steps:
1) A plurality of flexible delivery tube connects to the corresponding connection holes on the first connection part, respectively. The other end of each flexible delivery tube connects to a needle trocar that is inserted into the target body.
2) Achieving the alignment of one end of the wire output channel and the different connection holes or the quick connector of the flexible delivery tube on the first connection part via the movement of the first motion platform. Then, establishing the docking between one end of the wire output channel and the different connection holes or the quick connector of the flexible delivery tube on the first connection part via the back and forth movement of the first motion platform.
3) The wire driving mechanism drives the pushing wire to move back and forth along the wire output channel, which pushes the seed or seed strand forward. The seed or seed strand is set at the front of the pushing wire by the radioactive source feeding mechanism. Then the seed or seed strand is delivered through the flexible delivery tube and implanted into the target body via the needle trocar connected to the front end of the flexible delivery tube.

The radioactive source feeding mechanism adopts a magazine for supplying. The storage slot or storage holes in the magazine is set with a fourth elastic element that is used to push seeds or seed strands. The fourth elastic element exerts a certain thrust on the seeds or seed strands arranged in the storage slot or storage holes by its own elastic force. When the wire driving mechanism drives the pushing wire to retract to the rear of the magazine, the fourth elastic element pushes the seed or seed strand that is closest to the wire output channel within the storage slot or storage holes into the wire output channel, positioning the seed or seed strand in front of the pushing wire. Then the wire driving mechanism drives the pushing wire to push the seed or seed strand in front of the pushing wire forward. The seed or seed strand is pushed out of the magazine. Subsequently, the wire driving mechanism drives the pushing wire to reset. The fourth elastic element uses its own elastic force to push the seed or seed strand that is closest to the wire output channel within the storage slot or storage holes into the wire output channel, positioning the seed or seed strand in front of the pushing wire. Subsequently, pushing the seed or seed strand out of the magazine. This operation is repeated until several seeds or seed strands have accumulated in front of the pushing wire.

As illustrated in FIG. 19 and FIG. 23, once, in step (2), one end of the wire output channel connects to different connection holes on the first connection part or the quick connector of the flexible delivery tube, the wire driving mechanism drives the pushing wire to push the accumulated seeds or seed strands in front of the pushing wire forward. It makes the seeds or seed strands to pass through the wire output channel, the flexible delivery tube, and the needle trocar, and to be directly implanted into the target body, completing the implantation operation. During the implantation, as the seeds or seed strands are being implanted, the needle trocar 101504 is pulled outward partly from the human body. Each time the needle trocar 101504 is retracted by a certain distance, the flexible wire driving mechanism 24012 continues to push out the next seed 101505, working back and forth until the implantation of a plurality of seeds or seed strands is completed. The seeds or seed strands are implanted evenly and at intervals into the target body.

At the front end of the needle trocar 101504, there is a resistance section designed to hold the accumulated seeds or seed strands within the needle trocar, preventing them from slipping out directly from the front end of the needle trocar under the effect of gravity. Under the push of the pushing wire, the outermost seed or seed strand overcomes the resistance of the resistance section and is pushed out from the front end of the needle trocar. The implantation action is coordinated with the needle retraction action, allowing for the output of different seeds or seed strands to different depths of the target body.

The resistance section is the constricted part at the front end of the needle trocar, where the inner diameter of the constricted part is smaller than the outer diameter of the seeds, preventing the seeds or seed strands from falling directly from the front end of the needle trocar under the effect of gravity. Moreover, there is a slot along the side of the constricted part, which is parallel to the axis of the needle trocar, which is used to increase the elasticity of the constricted part and reduce the resistance of the pushing wire.

The described embodiments are only preferred embodiments of the present invention, and it should be noted that various alterations and improvements may be made therein by those of ordinary skill in the art without departing from the principle of the present invention and should also fall within the scope of protection of the present invention.

Although preferred embodiments have been described herein in detail, it should be noted and will be appreciated by those skilled in the art that numerous variations may be made within the scope of this invention without departing from the principle of this invention and without sacrificing its chief advantages. The terms and expressions have been used herein as terms of description and not terms of limitation. There is no intention to use the terms or expressions to exclude any equivalents of features shown and described or portions thereof and this invention should be defined in accordance with the claims which follow.

We claim:

1. A multi-channel radioactive source implantation device, comprising:
a first motion platform being used to control the relative position in space between one end of the wire output channel and the first connecting portion;
a first connecting portion;
a flexible pushing wire;
a wire driving mechanism connecting with the wire output channel and being used to drive the flexible pushing wire to move back and forth along the wire output channel;
a radioactive source feeding mechanism being used to set the radioactive source in front of the flexible pushing wire; and
a wire output channel being a rigid structure or a flexible structure that can be bent;
Wherein one end of the wire output channel and the first connecting portion are respectively located on the opposite sides of the first motion platform.

2. The multi-channel radioactive source implantation device according to claim 1, wherein the first connecting portion connects to the first connection part, and the first connection part has a plurality of connection holes. One end of the flexible delivery tube connects on the corresponding connection hole, and at the other end of the flexible delivery tube is connected to a needle trocar. The first motion platform is used to achieve relative motion in space between one end of the wire output channel and the first connection part, so that it makes the wire output channel connects with each flexible delivery tube on the first connection part to form a delivery channel for the radioactive source, achieving multi-channel implantation. The first connecting portion is one or a combination of an adhesive connecting portion, a welding connecting portion, a threaded connecting portion, a snap-fit connecting portion, or a latch connecting portion.

3. The multi-channel radioactive source implantation device according to claim 2, wherein the first motion platform is one of the following modes:
A. The first connection part moves and one end of the wire output channel remains stationary.
B. The first connection part remains stationary and one end of the wire output channel moves.
C. The first connection part moves and one end of the wire output channel also moves.
The first motion platform is used to achieve the relative movement of at least two degrees of freedom between the first connection part and one end of the wire output channel. The mode of relative movement is one of the following:
A. The first connection part is stationary and one end of the wire output channel performs motion within a plane.
B. The first connection part performs linear motion back and forth and one end of the wire output channel performs motion within a plane.
C. The first connection part performs motion within a plane and one end of the wire output channel performs linear motion back and forth.
D. The first connection part performs linear motion back and forth and motion within a plane and one end of the wire output channel is stationary.
The motion within a plane is one of the following types: single rotational movement, a rotational movement combined with a radial linear movement, double-joint rotational movement, or two linear movements along the XY axes.

4. The multi-channel radioactive source implantation device according to claim 3, wherein the first motion platform includes a back and forth motion module, a rotation motion module, and a radial motion module. One end of the wire output channel and/or the first connection part achieves three degrees of freedom of movement in space via the rotational movement in one direction and the linear movements in two directions of the first motion platform.
Alternatively, the first motion platform includes a back and forth motion module, a left-and-right motion module, and an up-and-down motion module. One end of the wire output channel and/or the first connection part achieves three degrees of freedom of movement in space via the linear movements in three directions of the first motion platform.
Alternatively, the first motion platform is a multi-joint robotic arm, which can drive one end of the wire output channel and/or the first connection part to freely move and position in three-dimensional space.
Alternatively, the first motion platform includes a back and forth motion module, a rotation motion module. One end of the wire output channel and/or the first connection part achieves movement in space via the rotational movement in one direction and the linear movements in one direction of the first motion platform.

5. The multi-channel radioactive source implantation device according to claim 4, wherein the first motion platform is installed at the exterior of the main housing or within the interior of the main housing

6. The multi-channel radioactive source implantation device according to claim 5, wherein when the first motion platform is installed inside the main housing and includes a back and forth motion module and a rotation motion module, the front side of the main housing is the first connecting portion, on which the first connection part is mounted. The first motion platform is designed in a drum-like shape, with the wire driving mechanism installed inside the drum. The rotation motion module drives the drum to rotate around its axis, and the back and forth motion module is installed inside the drum, used to drive the one end of the wire output channel to move back and forth.

7. The multi-channel radioactive source implantation device according to claim 6, a combination of a wire driving mechanism and a docking tube or a combination of a wire driving mechanism, a magazine base, and a docking tube is installed in the first motion platform. The installation of the magazine base and docking tube is perpendicular to the first connecting portion. A magazine is mounted on the wire driving mechanism or on the magazine base.
When the first motion platform is installed with a wire driving mechanism and a magazine base, the wire driving mechanism and the magazine base are connected via a flexible tube. In this case, the flexible connecting tube is a part of the wire output channel, and the outlet of the magazine base is the end of the wire output channel.
When the first motion platform is installed with a wire driving mechanism and a docking tube, the wire driving mechanism and the docking tube are connected via a flexible connecting tube. In this case, the flexible connecting tube is a part of the wire output channel, and the outlet of the docking tube is the end of the wire output channel.

8. The multi-channel radioactive source implantation device according to claim 2, wherein the flexible delivery tubes can be quickly installed onto the connection holes of the first connection part via quick connectors. There is a synchronized connector locking mechanism on the first connection part, which can simultaneously secure all the quick connectors mounted on the connection holes of the first connection part.

9. The multi-channel radioactive source implantation device according to claim 8, wherein the synchronized connector locking mechanism includes a connector clamping plate which is inside the first connection part. The connector clamping plate is equipped with connector clamping portions arranged in a array. In the unlocked state, all the connector clamping portions are staggered from the connection holes, allowing the quick connector of the flexible delivery tubes to be inserted into the connection holes without obstruction. During the locking process, the connector clamping portions are extended into the connection holes and inserted into the groove through the rotation or translation of the connector clamping plate driven by the clamping actuation mechanism, thereby securing all the quick connector of the flexible delivery tubes to the first connection part.

10. The multi-channel radioactive source implantation device according to claim 9, wherein the clamping actuation mechanism can be powered by electricity or manually operated, and it drives the rotation or translation of the connector clamping plate by a cam, a gear, or a belt drive.

11. The multi-channel radioactive source implantation device according to claim 9, wherein a flexible stylet is inside the flexible delivery tube, and it extends along the flexible delivery tube and fills the space of the needle trocar which is connected to the front end of the flexible delivery tube. This prevents blood from flowing into the needle trocar, otherwise blood coagulation can lead to the blockage of the needle trocar. The first connection part also has a synchronized stylet locking mechanism, it can simultaneously secure the flexible stylets which is inside each flexible delivery tube mounted on the first connection part.

12. The multi-channel radioactive source implantation device according to claim 11, wherein the synchronized stylet locking mechanism includes a stylet clamping plate which is inside the first connection part. The stylet clamping plate is equipped with stylet clamping portions arranged in a array. In the unlocked state, all stylet clamping portions are staggered with the connection holes, allowing the quick connector of the flexible delivery tube to be inserted through them. When locking the flexible stylet, a stylet clamping driving mechanism drives the stylet clamping plate to rotate or translate, misaligning the stylet clamping portions with the connection holes. The side of the quick connector of the flexible delivery tube has an opening that is connected to the internal elastic portion. The inside of the elastic portion adjacent to the flexible stylet inside this flexible delivery tube. As the stylet clamping plate rotates or translates, the stylet clamping portion presses against the elastic portion, so as to compress the flexible stylet through the elastic portion, achieving the flexible stylet locking.
Alternatively, the synchronized connector locking mechanism is the synchronized stylet locking mechanism. The connector clamping plate is the stylet clamping plate. The connector clamping portion is the stylet clamping portion. And the clamping driving mechanism is the stylet clamping driving mechanism. And the grooves of the quick connector is the opening that is connected to the internal elastic portion. When the clamping driving mechanism drives the connector clamping plate to rotate by a first angle or to translate by a first distance, the connector clamping portion inserts into the grooves of the quick connector of the flexible delivery tube without pressing against the elastic body. This action secures all the quick connector of the flexible delivery tubes to the first connection part, only achieving simultaneous locking of the quick connectors of all flexible delivery tubes, without simultaneously locking the flexible stylet inside the flexible delivery tubes.
When the clamping driving mechanism drives the connector clamping plate to rotate by a second angle or to translate by a second distance, the connector clamping plate presses against the elastic body during the rotation or translation process. The flexible stylet is compressed by the elastic body, it achieves the simultaneous locking of both the quick connector and the flexible stylet inside the flexible delivery tube.

13. The multi-channel radioactive source implantation device according to claim 11, wherein before implantation, the flexible stylet must be pulled out. The flexible stylet is a flexible wire with elasticity, it can be bent under the effect of an external force and return to its straight state after the external force is removed. The length of the flexible stylet is greater than 600 mm. The rear end of the flexible stylet extends backward from the rear end of the flexible delivery tube. The distance between the front end of the flexible stylet and the front end of the needle trocar connected to the front end of the flexible delivery tube is not exceeding 5 mm.

14. The multi-channel radioactive source implantation device according to claim 7, wherein when the first motion platform is equipped with a wire driving mechanism and a docking tube, the first motion platform is used to achieve relative motion in space of the docking tube and the first connection part, allowing the docking tube to dock with one of the flexible delivery tubes on the first connection part. The docking tube includes a docking head and a first floating connecting mechanism. The docking head is mounted on the first motion platform via the first floating connecting mechanism which enables the docking head to float within a range.

15. The multi-channel radioactive source implantation device according to claim 14, wherein the first floating connecting mechanism adopts two rotational joint modules.
Alternatively, the first floating connecting mechanism adopts one rotational joint module and one translational module.
Alternatively, the first floating connecting mechanism adopts two translational modules, with the movement paths of the two translational modules being orthogonal to each other or at a certain angle.
Alternatively, the first floating connecting mechanism adopts a supporting base with an elastic ball hinge. The elastic ball hinge consists of a third elastic element and a hinge ball. The docking head is installed at the hinge ball which allows the docking head to pivot through a certain angle. The third elastic element is used to provide elastic force to reset the docking head when the docking head has been deflected.

16. The multi-channel radioactive source implantation device according to claim 15, wherein when the first floating connecting mechanism adopts two rotational joint modules, the first floating connecting mechanism includes a first link, a second link, and a link base. The link base is mounted on the first motion platform. The second link is rotatably set on the link base through a first rotating axis and achieves the rotational resetting via the first elastic element. The first link is rotatably set on the second link through a second rotating axis and achieves the rotational resetting via the second elastic element. The docking head is set on the first link, or the docking head connects to the first link through a quick assembly and disassembly structure. The first rotating axis is parallel to the second rotating axis.

17. The multi-channel radioactive source implantation device according to claim 16, wherein the second elastic element is the first elastic element. One end of the first elastic element connects to the link base, and the other end connects to the first link. While the first elastic element facilitates the rotational resetting of the first link, it also enables the rotational resetting of the second link.

18. The multi-channel radioactive source implantation device according to claim 1, wherein the radioactive source feeding mechanism acts as a cutting mechanism. The pushing wire itself is a seed strand or a seed strand casing, or the front half of the pushing wire is a seed strand or seed strand casing, and the rear half of the pushing wire is an elastic wire. The seed strand and seed strand casing can be cut by the cutting mechanism, and the target length of the seed strand or seed strand casing is severed from the front end of the pushing wire by the cutting mechanism, thereby achieving the feeding of the seed strand or seed strand casing. When the severed part is a seed strand casing, the radioactive source feeding mechanism also includes a seed embedding mechanism. This seed embedding mechanism is capable of inserting seeds and/or spacers into the seed strand casing from one end or the side, to form a complete seed strand. The cutting mechanism is positioned at any location along the wire output channel.
Alternatively, the radioactive source feeding mechanism uses a magazine for feeding. The radioactive source feeding mechanism is directly integrated into the wire output channel. Seeds, or seed strands, or seed strand casings are loaded into the storage slot or storage holes within the magazine. The feeding mechanism installed on the magazine is used to place seeds, or seed strands, or seed strand casings at the front end of the pushing wire for feeding. When the magazine is loaded with a seed strand casing, the radioactive source feeding mechanism also includes a seed embedding mechanism. This seed embedding mechanism is capable of inserting seeds and/or spacers into the seed strand casing from one end or the side, forming a complete seed strand.
Alternatively, the radioactive source feeding mechanism uses a seed strand driving mechanism for feeding seed strands. The radioactive source feeding mechanism comprises a seed strand driving mechanism, a seed strand output channel, and a cutting mechanism. It outputs seed strands or seed strand casings by the seed strand driving mechanism and cuts them to the desired length by the cutting mechanism, to achieve the feeding of seed strands or seed strand casings. When the seed strand driving mechanism outputs a seed strand casing, the radioactive source feeding mechanism also includes a seed embedding mechanism. This seed embedding mechanism is capable of inserting seeds and/or spacers into the seed strand casing from one end or the side, thereby forming a complete seed strand. The seed strand driving mechanism is connected to the seed strand output channel. The seed strand output channel is a rigid structure or a flexible structure that can be bent. The seed strand output channel is connected with a divided channel or a channel switching mechanism. And the divided channel or the channel switching mechanism is also connected to the wire output channel.

19. The use method of the multi-channel radioactive source implantation device, wherein including the following steps:
1) Connect a plurality of flexible delivery tubes to the corresponding connection holes on the first connection part, respectively. The other end of each flexible delivery tube connects to a needle trocar that is inserted into a target body.
2) Achieving the aligning of one end of the wire output channel with the different quick connector of the flexible delivery tube via the movement of the first motion platform. Then, establishing the docking between one end of the wire output channel and the quick connector of the flexible delivery tube via the back and forth movement of the first motion platform.
3) The wire driving mechanism drives the pushing wire to move back and forth along the wire output channel, which pushes the seed or seed strand forward. The seed or seed strand is set at the front end of the pushing wire by the radioactive source feeding mechanism. Then the seed or seed strand is delivered through the flexible delivery tube and implanted into the target body via the needle trocar connected at the front end of the flexible delivery tube.

20. The use method of the multi-channel radioactive source implantation device according to claim 19, wherein the radioactive source feeding mechanism adopts a magazine for supplying. The seeds or seed strand is set in the storage slot or storage holes of the magazine. The magazine is set with a fourth elastic element that pushes seeds or seed strands to the seed output channel that is connected with the wire output channel. When the wire driving mechanism drives the pushing wire to retract behind the magazine, the fourth elastic element pushes the seed or seed strand that is closest to the seed output channel within the storage slot or storage holes into the seed output channel, setting the seed or seed strand in front of the pushing wire. Then the wire driving mechanism drives the pushing wire to push the seed or seed strand in front of the pushing wire forward. The seed or seed strand is pushed out of the magazine. Subsequently, the wire driving mechanism drives the pushing wire to retract. The fourth elastic element uses its own elastic force to push the seed or seed strand that is closest to the wire output channel within the storage slot or storage holes into the wire output channel, positioning the seed or seed strand in front of the pushing wire. Subsequently, pushing the seed or seed strand out of the magazine. This operation is repeated until several seeds or seed strands have accumulated in front of the pushing wire.
Once, in step (2), one end of the wire output channel docks to one of the quick connectors of the flexible delivery tubes, the wire driving mechanism drives the pushing wire to push the accumulated seeds or seed strands in front of the pushing wire forward. It makes the seeds or seed strands to pass through the wire output channel, the flexible delivery tube, and the needle trocar, and to be implanted respectively into the target body, completing the implantation operation.
